(19) United States
(12) Patent Application Publication
(10) Pub. No.: US 2025/0093331 A1
(43) Pub. Date: Mar. 20, 2025

(54) METHOD FOR DETERMINING ACUTE TOXICITY AND A SYSTEM USING SAID METHOD

(71) Applicant: City University of Hong Kong, Kowloon (HK)

(72) Inventors: Wenxiong WANG, Kowloon (HK); Yiteng XIA, Kowloon (HK)

(21) Appl. No.: 18/431,056

(22) Filed: Feb. 2, 2024

Related U.S. Application Data
(60) Provisional application No. 63/583,904, filed on Sep. 20, 2023.

(51) Int. Cl. G01N 33/50; A61K 49/00; G01N 21/64

(52) U.S. Cl. CPC G01N 33/5014; A61K 49/0008; G01N 21/6428; G01N 2021/6439; G01N 2333/4603

(57) ABSTRACT

A method for determining acute toxicity of an analyte includes the steps of: a) exposing a first fish cell culture to a first reference solution; b) incubating the first fish cell culture in step a) with a fluorescent probe; c) obtaining a first fluorescence parameter from the stained first fish cell culture; d) establishing a first linear regression relationship model between the first fluorescence parameter and viability of an animal model; and e) determining acute toxicity of the analyte by adopting the first linear regression relationship model. A system using the method for determining acute toxicity of an analyte is also addressed.

### TECHNICAL FIELD

The present invention relates to a method for determining acute toxicity of an analyte for example particularly, but not exclusively, a sewage effluent sample; and a system for determining acute toxicity of an analyte using the method.

### BACKGROUND OF THE INVENTION

Traditionally, acute toxicity testing requires substantial numbers of animals and uses death as an apical end point which requires large number of experimental animals and takes days to obtain the results. For example, in one testing process, it may require a substantial number of testing animals (about 60˜80) and takes two working days and uses death as integrative but crude end point. In addition, it is reported that in Europe alone, 2.56 million of fish were used in 2019 for research and testing purposes. It is appreciated that such a large number of experimental fish not only brings extra cost but also causes ethical issues. Apart from the experimental animals, the required volume of testing samples (˜500 L) and sewage effluents (˜1500 L) after testing are enormous which brings extra environmental issues.

Whilst there are reports of development and/or use of alternative methods for determining acute toxicity, particularly with the use of fish cell line in replacement of the conventional testing methods, it is believed that cell-based assay may show several orders of magnitude less sensitive than the animal-based results.

The invention seeks to eliminate or at least to mitigate such shortcomings, particularly by providing a new or otherwise improved cell-based assay with the combination of fluorescence technique and bioimaging tool for evaluating acute toxicity.

### SUMMARY OF THE INVENTION

In a first aspect of the present invention, there is provided a method for determining acute toxicity of an analyte, comprising the steps of: a) exposing a first fish cell culture to a first reference solution; b) incubating the first fish cell culture in step a) with a fluorescent probe; c) obtaining a first fluorescence parameter from the stained first fish cell culture; d) establishing a first linear regression relationship model between the first fluorescence parameter and viability of an animal model; and e) determining acute toxicity of the analyte by adopting the first linear regression relationship model.

In an optional embodiment, the first fish cell culture comprises a fish fin cell line isolated from grey rabbit fish (Siganus fuscescens).

Optionally, step a) comprises the step of collecting the first reference solution from a reference site at a first time point.

In an optional embodiment, the first reference solution comprises a 24-h flow-weighted composite effluent from a sewage effluent treatment work.

It is optional that the first reference solution has a concentration of pollutant by volume selected from any one of 0%, 6.5%, 12.5%, 25%, 50%, and 100%.

Optionally, step b) comprises the step of incubating a mixture of the first fish cell culture and the fluorescent probe in the dark.

It is optional that the fluorescent probe is selected from the group consisting of lysosomal tracker, mitochondrial tracker, (Z)-3-(4-(4-methylpiperazin-1-yl)phenyl)-2-(4-(pyridin-4-yl)phenyl) acrylonitrile (CSMPP), 7-ethoxyresorufin, H₂DCFDA, Fluo-4, AM, ThiolTracker, and a combination thereof.

In an optional embodiment, step c) comprises the steps of: taking confocal microscopy images of the stained first fish cell culture; and obtaining the first fluorescence parameter of the stained first fish cell culture from the confocal microscopy images.

Optionally, step c) further comprises step c1) obtaining fluorescence intensity of the stained first fish cell culture by way of a fluorescence plate reader.

It is optional that the fluorescence parameter corresponds to the fluorescent probe and is selected from the group consisting of lysosomal number, mitochondrial size, lysosomal pH, EROD activity, ROS production, Ca²⁺influx, GSH formation and a combination thereof.

Optionally, step d) comprises the steps of: incubating the animal model with the first reference solution for at least 12 h; determining the viability of the animal model; and building a first linear regression equation between the first fluorescence parameter and the viability of the animal model and obtaining an R-squared (R²) value therefrom.

In an optional embodiment, the animal model comprises any one of amphipod (Melita longidactyla), barnacle larvae (Balanus amphitrite), and shrimp (Metapenaeus ensis).

It is optional that the first reference solution has a concentration of pollutant by volume selected from any one of 0%, 6.5%, 12.5%, 25%, 50%, and 100%.

Optionally, the R-squared value is at least about 0.70.

In an optional embodiment, the method further comprises the steps of: a′) exposing a second fish cell culture to the analyte; b′) incubating the second fish cell culture in step a′) with a fluorescent probe; and c′) obtaining a second fluorescence parameter from the stained second fish cell culture.

Optionally, step e) comprises the steps of: determining, from the first linear regression relationship model, a reference value at 90% viability; and comparing the second fluorescence parameter with the reference value at 90% viability to determine the acute toxicity of the analyte.

It is optional that step e) further comprises the steps of: determining, from the first linear regression relationship model, a reference value at 50% viability; and comparing the second fluorescence parameter with the reference value at 50% viability to determine the acute toxicity of the analyte.

In an optional embodiment, the analyte comprises sewage effluent.

In an optional embodiment, step a) is repeated using a second and a third reference solution from the reference site at a second and third time point respectively.

Optionally, the first, second, and third time points are each different by a three-month interval.

In a second aspect of the present invention, there is provided a system for determining acute toxicity of an analyte in accordance with the method in accordance with the first aspect. The system comprises: a first fish cell culture stained with a fluorescent probe; a first reference solution in which the first fish cell culture is incubated; and a first linear regression relationship model established from a first fluorescence parameter obtained from the stained first fish cell culture and viability of an animal model; wherein the first linear regression relationship model contains acute toxicity of the analyte.

### BRIEF DESCRIPTION OF DRAWINGS

The patent or application file contains at least one drawing executed in color. Copies of this patent or patent application publication with color drawing(s) will be provided by the Office upon request and payment of the necessary fee.

The invention will now be more particularly described, by way of example only, with reference to the accompanying drawings, in which:

FIG. 1 shows the calibration curve of ICP-MS intensity of metal ions. The CPS (count per second) value of metal ions from ICP-MS was compared with the metal ion concentrations added. The equation of liner phase and R-squared value were shown as well;

FIG. 2 is a table summarizing the fluorescent probes used for staining, the target organelles, working concentrations, staining duration, and excitation/emission wavelength of the probes;

FIG. 3A shows the amounts of metal ions in the sewage samples measured via ICP-MS. The values are expressed as the absolute concentration of each metal ions in sewage samples (S1, S2, S3). Mean±SD (n=3);

FIG. 3B shows the amounts of organic pollutants measured via ICP-MS. The values are expressed as the absolute concentration of each metal ions in sewage samples. Mean±SD (n=3);

FIG. 4 shows the LC₅₀ value of experimental animals under different sewage exposure. The values are expressed as the rate of sewage (v/v). Mean±SD (n=4);

FIG. 5A shows the cell viability of RFF cells under 24- and 48-h exposure to different sewage contents. The cell viability was measured via MTT assay. The values are expressed as % of control, as compared with control reading (as 100%, no sewage added). Mean±SD (n=6) * p<0.05; ** p<0.01;

FIG. 5B shows the cell viability of RFM cells under 24- and 48-h exposure to different sewage contents. The cell viability was measured via MTT assay. The values are expressed as % of control, as compared with control reading (as 100%, no sewage added). Mean±SD (n=6) * p<0.05; ** p<0.01;

FIG. 5C shows the cell viability of ZF4 cells under 24- and 48-h exposure to different sewage contents. The cell viability was measured via MTT assay. The values are expressed as % of control, as compared with control reading (as 100%, no sewage added). Mean±SD (n=6) * p<0.05; ** p<0.01;

FIG. 6A shows RFF cells after 24-h exposure were stained with lysosomal tracker (yellow) and mitochondrial tracker (red). The graphs were taken by LSM900 confocal microscope and the representative graphs are shown;

FIG. 6B shows the calibration curve between lysosomal number and sewage effluent content. Lysosomal numbers after sewage effluents exposure were obtained from confocal graphs by ImageJ analysis. Mean±SD (n=6). Statistical comparison was made with the 0 group (no sewage effluents added); * p<0.05; ** p<0.01. The equation of liner phase and R-squared value are shown as well;

FIG. 6C shows the calibration curve between mitochondrial size and sewage effluent content. Mitochondrial size after sewage effluents exposure were obtained from confocal graphs by ImageJ analysis. Mean±SD (n=6). Statistical comparison was made with the 0 group (no sewage effluents added); * p<0.05; ** p<0.01. The equation of liner phase and R-squared value are shown as well;

FIG. 7A shows the RFF cells after 24-hours exposure were stained with lysosomal tracker (white) and CMSPP (green and red). The graphs were taken by LSM900 confocal microscope and the representative graphs are shown;

FIG. 7B shows the changes in lysosomal pH under different concentrations of sewage effluents after 24-h exposure. The results were obtained from confocal graphs above and analysed by ImageJ analysis. Mean±SD (n=6). Statistical comparison was made with the 0 group (no sewage effluents added); * p<0.05; ** p<0.01. The equation of liner phase and R-squared value are shown as well;

FIG. 8A shows the ratio of fluorescent intensity of two emission channels (615 nm (red), 503 nm (green)) was gained via Image J software and named as “R”. The ratio of fluorescent intensity under pH at 2.6 and 6.8 were named as Rₘₐₓ and Rₘᵢₙ respectively;

FIG. 8B shows that the pH value was compared with log [(R−Rₘᵢₙ)/(Rₘₐₓ−R)] and showed a linear relationship. The equation of liner phase and R-squared value were shown as well;

FIG. 9A shows the RFF cells after 24-hours exposure were stained with 7-ethoxyresorufin;

FIG. 9B shows the calibration curve between ERDO activity and sewage effluents content. The ERDO activity was obtained from confocal graphs corresponding to FIG. 9A by ImageJ analysis after sewage effluents exposure. Mean±SD (n=4). Statistical comparison was made with the 0 group (no sewage effluents added); * p<0.05; ** p<0.01. The equation of liner phase and R-squared value were shown as well;

FIG. 9C shows the RFF cells after 24-hours exposure were stained DC-FHDA;

FIG. 9D shows the calibration curve between ROS formation and sewage effluents content. The ROS formation was obtained from confocal graphs corresponding to FIG. 9C by ImageJ analysis after sewage effluents exposure. Mean±SD (n=4). Statistical comparison was made with the 0 group (no sewage effluents added); * p<0.05; ** p<0.01. The equation of liner phase and R-squared value were shown as well;

FIG. 9E shows the RFF cells after 24-hours exposure were stained with Fluo-4;

FIG. 9F shows the calibration curve between Ca²⁺ influx and sewage effluents content. The Ca²⁺ influx was obtained from confocal graphs corresponding to FIG. 9E by ImageJ analysis after sewage effluents exposure. Mean±SD (n=4). Statistical comparison was made with the 0 group (no sewage effluents added); * p<0.05; ** p<0.01. The equation of liner phase and R-squared value were shown as well;

FIG. 9G shows the RFF cells after 24-hours exposure were stained with ThiolTracker™ Violet;

FIG. 9H shows the calibration curve between GSH content and sewage effluents content. The GSH content was obtained from confocal graphs corresponding to FIG. 9G by ImageJ analysis after sewage effluents exposure. Mean±SD (n=4). Statistical comparison was made with the 0 group (no sewage effluents added); * p<0.05; ** p<0.01. The equation of liner phase and R-squared value were shown as well;

FIG. 10A shows the celebration curve between cellular parameter (lysosomal number) gained from confocal microscopy and fluorescent plate reader. Mean±SD (n=3). The equation of liner phase and R-squared value were shown as well;

FIG. 10B shows the celebration curve between cellular parameter (mitochondrial size) gained from confocal microscopy and fluorescent plate reader. Mean±SD (n=3). The equation of liner phase and R-squared value were shown as well;

FIG. 10C shows the celebration curve between cellular parameter (EROD activity) gained from confocal microscopy and fluorescent plate reader. Mean±SD (n=3). The equation of liner phase and R-squared value were shown as well;

FIG. 10D shows the celebration curve between cellular parameter (ROS formation) gained from confocal microscopy and fluorescent plate reader. Mean±SD (n=3). The equation of liner phase and R-squared value were shown as well;

FIG. 10E shows the celebration curve between cellular parameter (Ca²⁺ influx) gained from confocal microscopy and fluorescent plate reader. Mean±SD (n=3). The equation of liner phase and R-squared value were shown as well;

FIG. 10F shows the celebration curve between cellular parameter (GSH content) gained from confocal microscopy and fluorescent plate reader. Mean±SD (n=3). The equation of liner phase and R-squared value were shown as well;

FIG. 11A shows the changes in cellular parameter (mitochondrial size) under different exposure durations. RFF cells were exposed under different concentrations of sewage effluents for 6, 12 and 24 hours. Then cells were harvest by trypsin and stained with fluorescent probes described above and the fluorescent intensity of various parameters were obtained by plater reader. The mitochondrial size was measured and normalized by cell number separately. The values are expressed as fold of change, as compared with basal reading (as 1, no sewage effluents added). Mean±SD (n=4);

FIG. 11B shows the changes in cellular parameter (EROD activity) under different exposure durations. RFF cells were exposed under different concentrations of sewage effluents for 6, 12 and 24 hours. Then cells were harvest by trypsin and stained with fluorescent probes described above and the fluorescent intensity of various parameters were obtained by plater reader. The mitochondrial size was measured and normalized by cell number separately. The values are expressed as fold of change, as compared with basal reading (as 1, no sewage effluents added). Mean±SD (n=4);

FIG. 11C shows the changes in cellular parameter (ROS formation) under different exposure durations. RFF cells were exposed under different concentrations of sewage effluents for 6, 12 and 24 hours. Then cells were harvest by trypsin and stained with fluorescent probes described above and the fluorescent intensity of various parameters were obtained by plater reader. The mitochondrial size was measured and normalized by cell number separately. The values are expressed as fold of change, as compared with basal reading (as 1, no sewage effluents added). Mean±SD (n=4);

FIG. 11D shows the changes in cellular parameter (Ca²⁺ influx) under different exposure durations. RFF cells were exposed under different concentrations of sewage effluents for 6, 12 and 24 hours. Then cells were harvest by trypsin and stained with fluorescent probes described above and the fluorescent intensity of various parameters were obtained by plater reader. The mitochondrial size was measured and normalized by cell number separately. The values are expressed as fold of change, as compared with basal reading (as 1, no sewage effluents added). Mean±SD (n=4);

FIG. 11E shows the changes in cellular parameter (Ca²⁺ influx) under different exposure durations. RFF cells were exposed under different concentrations of sewage effluents for 6, 12 and 24 hours. Then cells were harvest by trypsin and stained with fluorescent probes described above and the fluorescent intensity of various parameters were obtained by plater reader. The lysosomal pH was calculated based on the reported equation. Mean±SD (n=4);

FIG. 12A shows the celebration between mitochondrial size under 24 hours exposure and survival rate of testing animals (amphipod, barnacle and shrimp). The cellular parameters (obtained by confocal microscope) were compared with the survival rate (at 90% and 50%) of animals at the same sewage content shown in FIGS. 13A and 13B. The equation of liner phase and R-squared value were shown as well;

FIG. 12B shows the celebration between lysosomal number under 24 hours exposure and survival rate of testing animals (amphipod, barnacle and shrimp). The cellular parameters (obtained by confocal microscope) were compared with the survival rate (at 90% and 50%) of animals at the same sewage content shown in FIGS. 13A and 13B. The equation of liner phase and R-squared value were shown as well;

FIG. 12C shows the celebration between lysosomal number under 24 hours exposure and survival rate of testing animals (amphipod, barnacle and shrimp). The cellular parameters (obtained by confocal microscope) were compared with the survival rate (at 90% and 50%) of animals at the same sewage content shown in FIGS. 13A and 13B. The equation of liner phase and R-squared value were shown as well;

FIG. 12D shows the celebration between ROS formation under 24 hours exposure and survival rate of testing animals (amphipod, barnacle and shrimp). The cellular parameters (obtained by confocal microscope) were compared with the survival rate (at 90% and 50%) of animals at the same sewage content shown in FIGS. 13A and 13B. The equation of liner phase and R-squared value were shown as well;

FIG. 12E shows the celebration between Ca²⁺ influx under 24 hours exposure and survival rate of testing animals (amphipod, barnacle and shrimp). The cellular parameters (obtained by confocal microscope) were compared with the survival rate (at 90% and 50%) of animals at the same sewage content shown in FIGS. 13A and 13B. The equation of liner phase and R-squared value were shown as well;

FIG. 12F shows the celebration between GSH content under 24 hours exposure and survival rate of testing animals (amphipod, barnacle and shrimp). The cellular parameters (obtained by confocal microscope) were compared with the survival rate (at 90% and 50%) of animals at the same sewage content shown in FIGS. 13A and 13B. The equation of liner phase and R-squared value were shown as well;

FIG. 12G shows the celebration between lysosomal pH under 24 hours exposure and survival rate of testing animals (amphipod, barnacle and shrimp). The cellular parameters (obtained by confocal microscope) were compared with the survival rate (at 90% and 50%) of animals at the same sewage content shown in FIGS. 13A and 13B. The equation of liner phase and R-squared value were shown as well;

FIG. 12H shows the celebration between cell viability under 24 hours exposure and survival rate of testing animals (amphipod, barnacle and shrimp);

FIG. 13A is a table summarizing the calculated cellular parameters when the survival rate of the testing animal at 90%;

FIG. 13B is a table summarizing the calculated cellular parameters when the survival rate of the testing animal at 50%;

FIG. 14A shows the celebration between cell viability of RFF under 24- and 48-hours exposure and sewage effluents content. Mean±SD (n=6);

FIG. 14B shows celebration ship between cell viability of RFF under 24- and 48-hour exposure and animal survival rate. The equation of liner phase and R-squared value were shown as well;

FIG. 15A shows the viability of different animals (amphipods, barnacle and shrimp) under different concentrations of sewage samples S1 exposure for 48 hours corresponding to FIG. 14B;

FIG. 15B shows the viability of different animals (amphipods, barnacle and shrimp) under different concentrations of sewage samples S2 exposure for 48 hours corresponding to FIG. 14B;

FIG. 15C shows the viability of different animals (amphipods, barnacle and shrimp) under different concentrations of sewage samples S3 exposure for 48 hours corresponding to FIG. 14B;

FIG. 15D is a table summarizing the animal survival rate and fish cell survival rate under sewage samples S1 after 48 hours exposure corresponding to FIG. 15A. The values are expressed as the survival rate of animals or fish cell after exposure. Mean±SD (n=4);

FIG. 15E is a table summarizing the animal survival rate and fish cell survival rate under sewage samples S2 after 48 hours exposure corresponding to FIG. 15B. The values are expressed as the survival rate of animals or fish cell after exposure. Mean±SD (n=4);

FIG. 15F is a table summarizing the animal survival rate and fish cell survival rate under sewage samples S3 after 48 hours exposure corresponding to FIG. 15C. The values are expressed as the survival rate of animals or fish cell after exposure. Mean±SD (n=4);

FIG. 16A shows the celebration curve between cellular parameter (mitochondrial size, obtained by fluorescence plate reader) under 12 hours exposure and animal survival rate of testing animals (amphipod, barnacle, shrimp) The cellular parameters (obtained by fluorescence plate reader) were compared with the survival rate (at 90% and 50%) of animals at the same sewage content shown in FIGS. 17A and 17B. The equation of liner phase and R-squared value were shown as well;

FIG. 16B shows the celebration curve between cellular parameter (EROD activity, obtained by fluorescence plate reader) under 12 hours exposure and animal survival rate of testing animals (amphipod, barnacle, shrimp) The cellular parameters (obtained by fluorescence plate reader) were compared with the survival rate (at 90% and 50%) of animals at the same sewage content shown in FIGS. 17A and 17B. The equation of liner phase and R-squared value were shown as well;

FIG. 16C shows the celebration curve between cellular parameter (ROS formation, obtained by fluorescence plate reader) under 12 hours exposure and animal survival rate of testing animals (amphipod, barnacle, shrimp) The cellular parameters (obtained by fluorescence plate reader) were compared with the survival rate (at 90% and 50%) of animals at the same sewage content shown in FIGS. 17A and 17B. The equation of liner phase and R-squared value were shown as well;

FIG. 16D shows the celebration curve between cellular parameter (Ca²⁺ influx, obtained by fluorescence plate reader) under 12 hours exposure and animal survival rate of testing animals (amphipod, barnacle, shrimp) The cellular parameters (obtained by fluorescence plate reader) were compared with the survival rate (at 90% and 50%) of animals at the same sewage content shown in FIGS. 17A and 17B. The equation of liner phase and R-squared value were shown as well;

FIG. 16E shows the celebration curve between cellular parameter (lysosomal pH, obtained by fluorescence plate reader) under 12 hours exposure and animal survival rate of testing animals (amphipod, barnacle, shrimp) The cellular parameters (obtained by fluorescence plate reader) were compared with the survival rate (at 90% and 50%) of animals at the same sewage content shown in FIGS. 17A and 17B. The equation of liner phase and R-squared value were shown as well;

FIG. 17A is a table summarizing the calculated cellular parameters when the survival rate of the testing animal at 90%; and

FIG. 17B is a table summarizing the calculated cellular parameters when the survival rate of the testing animal at 50%.

### DETAILED DESCRIPTION OF OPTIONAL EMBODIMENT

As used herein, the forms “a”, “an”, and “the” are intended to include the singular and plural forms unless the context clearly indicates otherwise.

The words “example” or “exemplary” used in this invention are intended to serve as an example, instance, or illustration. Any aspect or design described in this disclosure as “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances.

As used herein, the phrase “about” is intended to refer to a value that is slightly deviated from the value stated herein. For example, “about 0.70” may be meant that any value from 0.68, 0.685, 0.69, 0.692 . . . 0.7, 0.705 . . . 0.71 . . . 0.718 . . . 0.72.

Without wishing to be bound by theory, the inventors have, through their own research, trials, and experiments, devised a cell-based assay which combines a fish cell line with fluorescent technology to reveal the subcellular toxicity of an analyte, in particular, sewage effluents. In particular, with the help of confocal microscope, changes in cellular organelles and enzyme activity could be visualized and quantified. In one example embodiment, it is found that among various cellular indexes, such as lysosomal number, mitochondrial size, lysosomal pH could sensitively respond to sewage effluents exposure. By comparing the cellular indexes with animal viability, suggesting that the present invention could represent the sewage content and/or toxicity more accurately. Furthermore, with the help of this cell-based assay, only 10 mL of exposure medium would be sufficient for a surrogate evaluation of the whole sewage effluent quality and/or toxicity determination and similar result could be achieved in a much shorter time. It is believed the present invention may be potentially extended to other applications such as providing preliminary toxicity result of drugs, cosmetics and additive, etc. It is also believed that the present invention may provide an access to replace, reduce or refine chronic animal tests with the characteristics of easy to follow, less time consuming and less expensive.

In a first aspect of the present invention, there is provided a method for determining acute toxicity of an analyte, comprising the steps of: a) exposing a first fish cell culture to a first reference solution; b) incubating the first fish cell culture in step a) with a fluorescent probe; c) obtaining a first fluorescence parameter from the stained first fish cell culture; d) establishing a first linear regression relationship model between the first fluorescence parameter and viability of an animal model; and e) determining acute toxicity of the analyte by adopting the first linear regression relationship model.

In an embodiment, the step a) may comprise the step of incubating the first fish culture with the first reference solution for at least 12 h, such as 12 h to 48 h, 12 h to 40 h, 12 h to 36 h, 12 h to 30 h, 12 h to 28 h, and in particular, 12 h to 24 h. It is believed that the incubation time varying between 12 h and 24 h would have no significant in the fluorescence parameters determined in the subsequent steps. Thus, it is believed that one may shorten the incubation time from 24 h to 12 h upon enabling the present invention for achieving a faster toxicity determination.

The first fish culture may comprise a plurality of fish cells seeded in, for example, a confocal dish, culture well/plate/dish and the like. The fish cells may be of a density of about 1×10⁶ cells per dish/well/plate. In an embodiment, the first fish culture may comprise any one of a fish fin cell line (RFF) isolated from grey rabbit fish (Siganus fuscescens), a macrophagic-like cell line (RFM) isolated from grey rabbit fish (Siganus fuscescens), and a fibroblast cell line (ZF4) isolated from zebrafish. In a preferred embodiment, the first fish culture may comprise RFF isolated from grey rabbit fish (Siganus fuscescens).

In an embodiment, step a) may further comprise the step of collecting the first reference solution from a reference site at a first time point. In particular, depending on the nature of the analyte, different (first) reference solution may be used. In an example embodiment, the analyte may comprise sewage effluent, and in this case, the first reference solution may be a composite effluent such as a 24-h flow-weighted composite effluent collected from a sewage effluent treatment work/plant. It is appreciated the effluent may be collected by way of suitable technical means.

Optionally or additionally, step a) may be repeated using a second and a third reference solution from the reference site such as the sewage treatment work as described herein at a second and a third time point respectively. In particular, the first, second, and third time points may be each different by a three-month interval. For example, if the first reference solution is collected from the reference site at a first time point of October 2022, then the second reference solution may be collected from the reference site at a second time point of January 2023, whereas the third reference solution may be collected from the references site at a third time point of April 2023.

The first fish culture may be exposed to the first reference solution having a concentration of pollutant by volume selected from any one of 0%, 6.5%, 12.5%, 25%, 50%, and 100%. In some embodiments, the pollutant may comprise heavy metals, organic pollutants and a combination thereof. In some embodiments, the heavy metals may include any one of arsenic, cadmium, cobalt, chromium, copper, manganese, lead, antimony, and zinc. In some embodiments, the organic pollutants may include any one of residual chlorine, bromoform, bromodichloromethane, chloroform, dibromochloromethane, methylene chloride, carbon tetrachloride, 1,1-dichloroethane, 1,2-dichloroethane, tetrachloroethylene, bromoacetic acid, chloroacetic acid, dibromoacetic acid, dichloroacetic acid, trichloroacetic acid, and 1,2,4-trichlorobenzene.

After exposing the first fish culture to the first reference solution in step a), in step b), the first fish culture may be incubated with a fluorescent probe. In particular, the fluorescent probe may be added to the first fish culture in step a) to form a mixture, followed by incubating the mixture in the dark. In some embodiments, the fluorescent probe may be selected from the group consisting of lysosomal tracker, mitochondrial tracker, (Z)-3-(4-(4-methylpiperazin-1-yl)phenyl)-2-(4-(pyridin-4-yl)phenyl) acrylonitrile (CSMPP), 7-ethoxyresorufin, H₂DCFDA, Fluo-4, AM, ThiolTracker, and a combination thereof. Based on the fluorescent probe used, the concentration as well as the incubation time may be adjusted according to actual needs and/or protocol. For example, in some embodiments where the fluorescent probe may be mitochondrial tracker such as MitoTracker™ Deep Red M22426, the first fish culture in step a) may be incubated with the mitochondrial tracker at a concentration of about 0.1 μM for about 15 min.

Step c) may include the steps of taking confocal microscopy images of the stained first fish cell culture; and obtaining the first fluorescence parameter of the stained first fish cell culture from the confocal microscopy images. In some embodiments, the stained first fish culture may be washed with, for example, 1× PBS prior to taking confocal images. The confocal images may be taken by confocal microscope with specific excitation and emission corresponding to the fluorescent probe used in step b). After that, the confocal images may be analyzed and quantified by specific software such as ImageJ software to obtain the first fluorescence parameter.

In some other embodiments, the first fluorescence parameter may be determined by way of a fluorescence plate reader instead of confocal microscopy. In these embodiments, step c) may further include step c1) obtaining fluorescence intensity of the stained first fish cell culture by way of a fluorescence plate reader. After obtaining the fluorescence intensity, it might be converted to normalized fluorescence intensity by dividing the fluorescence intensity with cell number. The normalized fluorescence intensity might be further converted to fold of change of fluorescence intensity by comparing with the fluorescence intensity of the first fish culture incubated with the first reference solution with 0% concentration of pollutant. In sum, in these embodiments, the first fluorescence parameter may be fold of change of fluorescence intensity as described herein.

In some embodiments, the first fluorescence parameter may correspond to the fluorescent probe such as the fluorescence intensity of the fluorescent probe determined from the confocal image as described herein and is selected from the group consisting of lysosomal number, mitochondrial size, lysosomal pH, EROD activity, ROS production, Ca²⁺ influx, GSH formation and a combination thereof.

In some embodiments where the first fluorescence parameter may be determined by way of fluorescence plate reader, the first fluorescence parameter may be the fold of change of fluorescence intensity corresponding to the fluorescent probe and is selected from the group consisting of lysosomal number, mitochondrial size, lysosomal pH, EROD activity, ROS production, Ca²⁺ influx, GSH formation and a combination thereof.

In step d), it may comprise the steps of: incubating the animal model with the first reference solution for at least 12 h; determining the viability of the animal model; and building a first linear regression equation between the first fluorescence parameter and the viability of the animal model and obtaining a R-squared (R²) value therefrom.

In some embodiments, the animal model may comprise any one of amphipod (Melita longidactyla), barnacle larvae (Balanus amphitrite), and shrimp (Metapenaeus ensis). One or more of these animal models may be incubated with the first reference solution having a concentration of pollutant by volume selected from any one of 0%, 6.5%, 12.5%, 25%, 50%, and 100% for at least 12 h, 12 h to 48 h, 18 h to 48 h, 20 h to 48 h, particularly 24 h to 48 h. After that, the viability of the animal model may be determined by calculating the number of survived animal models over the number of animal models exposing to the first reference solution with a concentration of pollutant by volume of 0%. The obtained viability of animal model may then be used for constructing a first linear regression model relationship with the first fluorescence parameter by plotting a graph of the viability of animal model against the first fluorescence parameter. By doing so, a first linear regression equation between the first fluorescence parameter and the viability of the animal model as well as an R-squared (R²) value may therefore obtained. In particular, the R² value may be at least about 0.70 such as from about 0.68 to about 0.98, from about 0.69 to about 0.98, from about 0.69 to about 0.97, from about 0.70 to about 0.97, and the like.

In some embodiments, step d) may be performed after completing steps a) to c). In some other embodiments, step d) may be performed in parallel to any one of steps a) to c).

The method of the present invention may further comprise the steps of: a′) exposing a second fish cell culture to the analyte such as sewage effluent as described herein; b′) incubating the second fish cell culture in step a′) with a fluorescent probe; and c′) obtaining a second fluorescence parameter from the stained second fish cell culture.

In some embodiments, the second fish cell culture may be any one of a fish fin cell line (RFF) isolated from grey rabbit fish (Siganus fuscescens), a macrophagic-like cell line (RFM) isolated from grey rabbit fish (Siganus fuscescens), and a fibroblast cell line (ZF4) isolated from zebrafish. Preferably, the second fish cell culture may be identical to the first fish culture used for obtaining the first fluorescence parameter, which may comprise RFF isolated from grey rabbit fish (Siganus fuscescens).

In particular, step a′) may be performed in a similar manner as step a), in which the second fish cell culture may be incubated with the analyte for at least 12 h, followed by incubating the second fish culture with a fluorescent probe as described herein. After that, the stained second fish culture may be arranged to obtain the second fluorescence parameter by way of confocal microscopy or fluorescence plate reader as described in step c).

In some embodiments, steps a′) to c′) may be performed after completing steps a) to d). In some other embodiments, steps a′) to c′) may be performed in parallel with any one of steps a) to d).

Turning now to step e), it may comprise the steps of determining, from the first linear regression relationship model, a reference value at 90% viability; and comparing the second fluorescence parameter with the reference value at 90% viability to determine the acute toxicity of the analyte. In particular, by using the first linear regression equation obtained in step d), one may fit the animal model viability into the equation to obtain the corresponding reference value.

In an embodiment, in a plot of animal model viability (y-axis) against a first fluorescence parameter such as mitochondrial size (x-axis) with a first linear regression equation such as y=2.36x−167.24, by putting the animal model viability of 90% into the equation, the first reference value (x value) will be determined as 71.25. Then, by comparing the second fluorescence parameter such as mitochondrial size determined in step c′) with this reference value, and if it is lower than such reference value, then it may be expected that the analyte (sewage effluent) could cause 10% death of the animal model and therefore could be considered as toxic.

In another embodiment, in a plot of animal model viability (y-axis) against a first fluorescence parameter such as Ca²⁺ influx (x-axis) with a first linear regression equation such as y=−0.69x+127.37, by putting the animal model viability of 90% into the equation, the first reference value (x value) will be determined as 183.3. Then, by comparing the second fluorescence parameter such as Ca²⁺ influx determined in step c′) with this reference value, and if it is higher than such reference value, then it may be expected that the analyte (sewage effluent) could cause 10% death of the animal model and therefore could be considered as toxic.

In an optional or additional embodiment, step e) may further comprise the steps of: determining, from the first linear regression relationship model, a reference value at 50% viability; and comparing the second fluorescence parameter with the reference value at 50% viability to determine the acute toxicity of the analyte. Similarly, in this embodiment, by using the first linear regression equation obtained in step d), one may fit the animal model viability (50% viability in this case) into the equation to obtain the corresponding reference value. For example, referring again to the first linear regression equation such as y=2.36x−167.24, at 50% animal model viability, the corresponding first reference value (x value) will be determined as 71.08, and by comparing the second fluorescence parameter such as mitochondrial size determined in step c′) with this reference value, and if it is lower than such reference value, then it may be expected that the analyte (sewage effluent) could cause 50% death of the animal model and therefore could be considered as toxic.

Similarly, referring again to the first linear regression equation such as y=−0.69x+127.37, at 50% animal model viability, the corresponding first reference value (x value) will be determined as 183.87, and by comparing the second fluorescence parameter such as mitochondrial size determined in step c′) with this reference value, and if it is higher than such reference value, then it may be expected that the analyte (sewage effluent) could cause 50% death of the animal model and therefore could be considered as toxic.

In a second aspect of the present invention, there is provided a system for determining acute toxicity of an analyte in accordance with the method as described herein. The system may comprise: a first fish cell culture stained with a fluorescent probe as described herein; a first reference solution as described herein in which the first fish cell culture is incubated; and a first linear regression relationship model established from a first fluorescence parameter obtained from the stained first fish cell culture and viability of an animal model as described herein; wherein the first linear regression relationship model contains acute toxicity of the analyte.

In some embodiments, the system may further comprise a second fish cell culture stained with the fluorescent probe which contains second fluorescence parameter as described herein.

Hereinafter, the present invention is described more specifically by way of examples, but the present invention is not limited thereto.

### EXAMPLES

### Materials and Methods

### Cell Culture

A keratinocyte cell line and macrophagic cell line isolated from rabbitfish (S. fuscescens) and a liver cell line isolated from zebrafish (Danio rerio). The fish fin (RFF) cells and macrophage (RFM) were cultured as reported. The full growth medium for RFF and RFM cells were the Dulbecco's modified Eagles medium (DMEM) supplemented with 100 IU/mL of penicillin, 100 μg/mL of streptomycin, 2 μg/mL of amphotericin-B, and 20% heat inactivated fetal bovine serum. The full growth medium for ZF4 cells were the Dulbecco's modified Eagles medium F-12 (DMEM F-12) supplemented with 100 IU/mL of penicillin, 100 μg/mL of streptomycin, 2 μg/mL of amphotericin-B, and 10% heat inactivated fetal bovine serum. All these cell lines were incubated at 28° C. in a water-saturated 5% CO₂ incubator. After 70% of confluence, cells were harvested with 1 mL of 0.25% trypsin for 3 min and trypsinization was stopped by adding 5 mL of full growth medium.

### Collection of Sewage Effluents

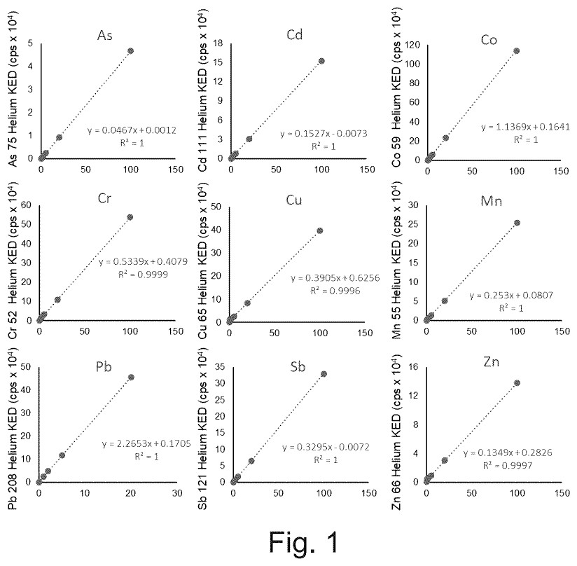

A 24-h flow-weighted composite effluent sample was collected from the sewage effluents treatment works in Hong Kong on three occasions (October 2022, January 2023, and April 2023), and named as S1, S2, S3 separately. Effluent samples were shipped immediately to the laboratory on the same day of collection. The salinity (% 0), dissolved oxygen (mg/L) and pH were determined. The metal pollutant concentrations were determined by inductively coupled plasma-mass spectrometry (ICP-MS). Briefly, 200 μL of sewage effluents were added into 200 μL 69% HNO₃ for digestion, and then diluted 100 times for ICP-MS (N8150060, Perkin Elmer) analysis. The standard curves of metals are shown in FIG. 1. For organic pollutants test, total residual chlorine was tested based on the APHA 4500 CI G method; bromoform, bromodichloromethane, chloroform, dibromochloromethane, methylene chloride, carbon tetrachloride, 1,1-dichloroethane, 1,2-dichloroethane, tetrachloroethlyene were tested based on USEPA 8260B method by GC-MS; bromoacetic acid, chloroacetic acid, dibromoacetic acid, dichloroacetic acid, trichloroacetic acid were tested based on TG-ENV-WW-79 by GC-ECD; 1,2,4-trichlorobenzene TG-ENV-WW-86 method by GC-MSD. Details of the GC-MS experiments, TG-ENV-WW-79, and TG-ENV-WW-86 are as follows:
TG-ENV-WW-79 based on METHOD 8121 from United States Environmental Protection Agency

The dual-column/dual-detector approach involves the use of two 30 m×0.53 mm ID fused-silica open-tubular columns of different polarities, thus different selectivities towards the target compounds. The columns are connected to an injection tee and two identical detectors.

Column 1—30 m×0.53 mm ID fused-silica open-tubular column, crosslinked and chemically bonded with 95 percent dimethyl and 5 percent diphenyl-polysiloxane (DB-5, RTx-5, SPB-5, or equivalent), 0.83 μm or 1.5 μm film thickness.

Column 2—30 m×0.53 mm ID fused-silica open-tubular column crosslinked and chemically bonded with 14 percent cyanopropylphenyl and 86 percent dimethyl-polysiloxane (DB-1701, RTx-1701, or equivalent), 1.0 μm film thickness.

Splitter: If the splitter approach to dual column injection is chosen, following are three suggested splitters. An equivalent splitter is acceptable. See Sec. 7.5.1 for a caution on the use of splitters.

Splitter 1—J&W Scientific press-fit Y-shaped glass 3-way union splitter (J&W Scientific, Catalog no. 705-0733).

Splitter 2—Supelco 8 in. glass injection tee, deactivated (Supelco, Catalog no. 2-3665M).

Splitter 3—Restek Y-shaped fused-silica connector (Restek, Catalog no. 20405).

Column rinsing kit (optional): Bonded-phase column rinse kit (J&W Scientific,

Catalog no. 430-3000 or equivalent).

Microsyringes—100 μL, 50 μL, 10 μL (Hamilton 701 N or equivalent), and 50 μL (Blunted, Hamilton 705SNR or equivalent). Balances-Analytical, 0.0001 g.

Volumetric flasks, Class A—10 mL to 1000 mL.

### TG-ENV-WW-86 Based on ISO 11423-1 Testing Method

The ISQ™ GC-MS was evaluated at a scanning speed of 2,650 u/see, which represents 10 full scan mass spectra taken per second (scanning m/z 35 to 300 in 0.1 sec). An OI Eclipse™ 4660 Purge-and-Trap Sample Concentrator equipped with a sample heater and 4551A autosampler were used to deliver 5 mL of sample for analysis. The internal standard and surrogates were added by the Standard Addition Module (SAM) unit. The calibration curve range was 0.4 μg/L to 40 μg/L in the sample. The on-column amount for each compound was 0.05 ng to 5 ng due to the 40 mL/min split flow during the injection.

### GC-MS Testing Process and Parameters

| | Conditions | |
|---|---|---|
| Purge and Trap Parameters | | |
| Sample volume | 5 | mL |
| Sample purge temperature | 40° | C. |
| Purge | 40 mL/min for 11 min | |
| Water management temperature: | 110° C., Desorb: 0° C., 240° C. | |
| Purge bake | | |
| Desorb preheat temperature | 180° | C. |
| Desorb temperature | 190° C. for 0.5 min | |
| Bake rinse cycles | twice | |
| Bake cycle | 210° C. for 10 min | |
| GC-MS parameters | | |
| Column | Thermo Scientific TRACE TR 524, | |
| | 20 m × 0.18 mm, 1.0 μm | |
| Inlet liner | P and T adapter kit | |
| Inlet temperature | 175° | C. |
| Split flow | 40 | mL/min |
| Column flow | 25 psi at constant pressure | |
| GC temperature program | 40° C. for 4 min, 18° C./min to | |
| | 100° C., 40° C./min to 230° C. | |
| | for 5 min | |
| Solvent delay | 0.5 min before activating filament | |
| MS source temperature | 250° | C. |

| | |
|---|---|
| Full scan | m/z 35 to 300 |
| Scan speed | 2650 u/sec (0.1 sec) |

The determination of benzene and some derivatives of the water sample is in accordance with Part 1: Headspace gas chromatographic method. ISO 11423-1, 1997.

### Cytotoxicity Study

Cell viability was determined by the MTT assay to quantify the cytotoxicity. Cells were seeded at a density of 2×10⁴ cells/well in 96-well plate and cultured for 12 h. Sewage effluent sample was mixed with the medium (DMEM) at various concentrations (6.5%, 12.5%, 25%, 50%, 100% v/v). After treatment, 0.5 mg/mL of 3-(4,5-Dimethylthiazol-2-yl)-2,5-Diphenyltetrazolium Bromide (MTT) solution was added, and dimethyl sulfoxide (DMSO) was added to dissolve the produced purple crystal. Absorbance was measured by a microplate reader at 570 nm.

### Visualization of Cellular Organelles and Lysosomal pH

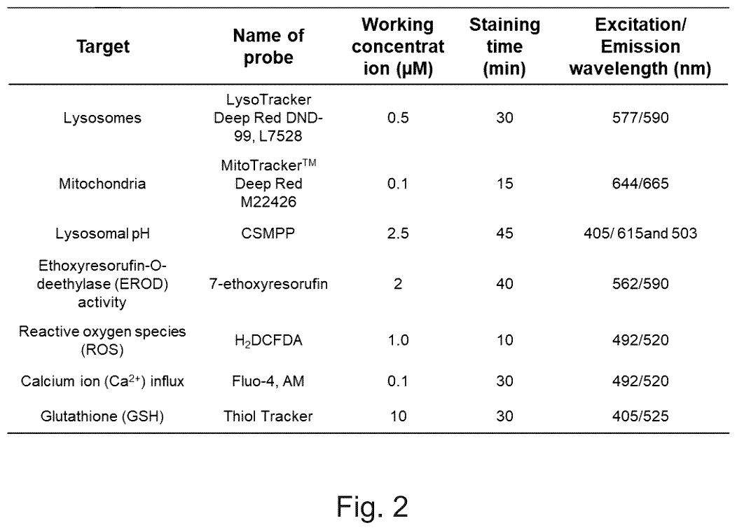

RFF cells were seeded in confocal dishes at a density of 1×10⁶ cells/dish and cultured for 12 h. Then the cells were exposed to medium with different sewage effluent samples at final concentrations of 6.5%, 12.5%, 25%, 50% and 100% (v/v) for 24 h. The cell organelles lysosomes and mitochondria were stained by 0.5 μM lysosomal tracker (LysoTracker Deep Red DND-99, L7528, Thermo Fisher) and 0.1 μM mitochondrial tracker (MitoTracker™ Deep Red M22426) for 30 min, respectively (FIG. 2).

To visualize the lysosomal pH, cyanostylbene derivative, (Z)-3-(4-(4-methylpiperazin-1-yl)phenyl)-2-(4-(pyridin-4-yl)phenyl) acrylonitrile (CSMPP) was added into culture medium at a final concentration of 2.5 μM and stained for 45 min (FIG. 2). After staining, 1× PBS was used to wash the residue probes, and the confocal images were collected by the confocal microscope LSM900 (Carl Zeiss). The fluorescent intensities of lysosomes and mitochondria were measured via microplate reader (FlexStation Multi-mode Microplate Reader) with this excitation and emission wavelength as described above. For lysosomal pH calculation, the ratio of fluorescent intensity of two emission channels (615 nm (red), 503 nm (green)) was gained via Image J software and named as “R”. The ratio of fluorescent intensity under pH at 2.6 and 6.8 were named as Rₘₐₓ and Rₘᵢₙ respectively. The pH value showed a linear relationship with log [(R−Rₘᵢₙ)/(Rₘₐₓ−R)]. The lysosomal pH was calculated according to the following equation:

$$pH = - log [ ( R - {R}_{min} ) / ( {R}_{max} - R ) ] - 2.9 \; 08 / ( - 0.6189 )$$

### Cellular Enzyme and Protein Measurements

In brief, RFF cells were seeded in confocal dishes. After overnight culture, the cells were exposed to medium with different sewage effluents concentrations. For ethoxyresorufin-O-deethylase (EROD) activity assay, 7-ethoxyresorufin was added to the cell medium after exposure at final concentration of 0.5 mg/L and incubated for 40 min (Table 1). The formation of reactive oxygen species (ROS) in RFF cells were visualized via H₂DCFDA (D399, Thermo Fisher). Briefly, RFF cells after exposure were incubated with 1 μM H₂DCFDA regent for 10 min. The influx of calcium ion (Ca) in cell were measured via Fluo-4, AM (F14201, Thermo Fisher). Briefly, RFF cells after exposure were incubated with 0.1 μM Fluo-4, AM regent for 30 min. The glutathione (GSH) content in cell were measured via ThiolTracker™ Violet (T10095, Thermo Fisher). RFF cells after exposure were incubated with 10 UM ThiolTracker™ Violet regent for 30 min. After staining, cells were then washed with 1×PBS twice to remove the solution. The confocal images were collected by the confocal microscope LSM900 (Carl Zeiss) with specific excitation and emission wavelength (λex=562/λem=590 nm for EROD, λex=492/λem=520 nm for ROS, λex=492/λem=520 nm for Ca²⁺ and λex=405/λem=590 nm for GSH) and quantified by ImageJ software afterwards. The fluorescent intensity of cell solution was measured via microplate reader (FlexStation Multi-mode Microplate Reader) with specific excitation and emission wavelength as described above. The specific EROD activity, ROS formation, Ca²⁺ influx and GSH content was calculated from the relative fluorescence units (RFU) as the product of resorufin and normalized with cell number. Summary of different probes for the staining purposes is given in FIG. 2.

### In Vivo Toxicity of Sewage Effluents

The animals used for sewage effluents toxicity test included amphipod (Melita longidactyla), barnacle larvae (Balanus amphitrite), and shrimp (Metapenaeus ensis). Amphipods were collected from local coastal waters from Sai Kung with body size around 0.5-0.7 cm. Experimental amphipods were acclimatized in fully aerated seawater (temperature: 22±1° C., salinity: 30% %) in the laboratory before test. Barnacle larvae at stage II were released from adult barnacles collected from Sai Kung. Experimental larvae were acclimatized in fully aerated seawater held in 500 mL glass beaker (temperature: 22±1° C., salinity: 30% %) and fed with diatom Chaetoceros gracilis for 24 h in laboratory before testing. After acclimation, experimental animals were exposed to sewage effluents at final concentrations of 6.5%, 12.5%, 25%, 50%, and 100% (v/v) for 48 h. The number of survived animals were calculated after the exposure.

### Statistical Analysis

All the data were analyzed using SPSS software. The results were expressed as mean+SD. One-way T-test was used to evaluate the difference between the control group and each experimental group (p<0.05).

### Example 1

### Pollutant's Index of Sewage Effluents and Whole Animal Toxicity

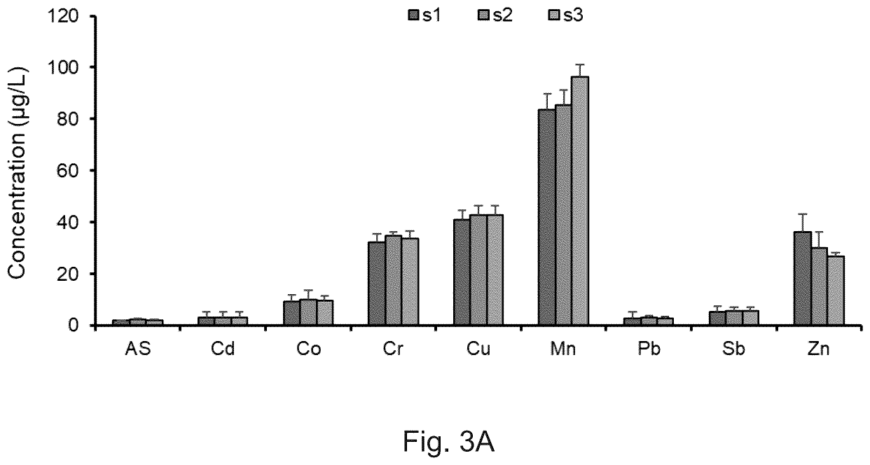

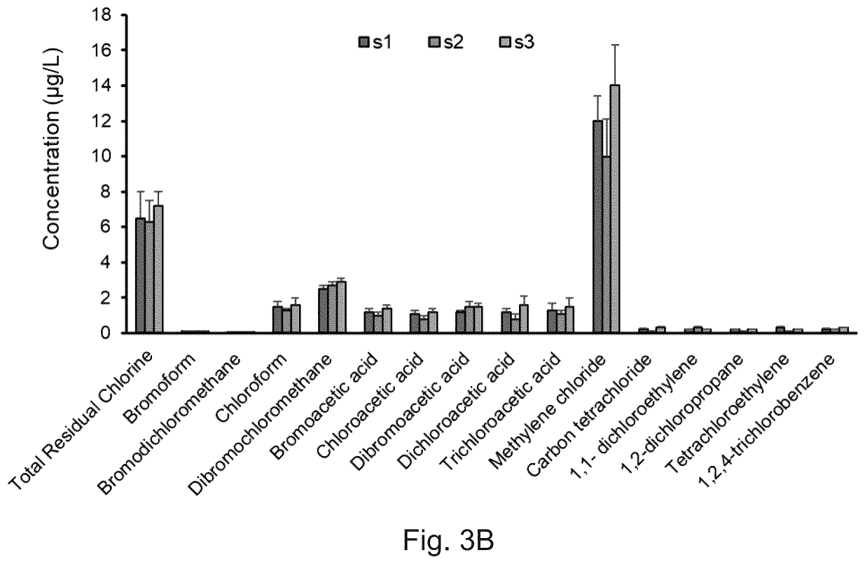

The salinity of all three sewage effluents samples was 20% %, dissolved oxygen was 6.8-7.0 mg/L, and the pH was 7.8-7.9. The metal concentrations detected in sewage effluents samples are shown in FIG. 3A. In all three sewage effluents samples, Mn was the most abundant metal followed by Cu and Zn. Other pollutants in sewage samples were quantified and shown in FIG. 3B. Among the tested pollutants, methylene chloride was the most abundant one (10˜13 μg/L), follow with residual chloride (˜6 μg/L). The content of all organic pollutants in sewage samples were lower than the limits in Hong Kong.

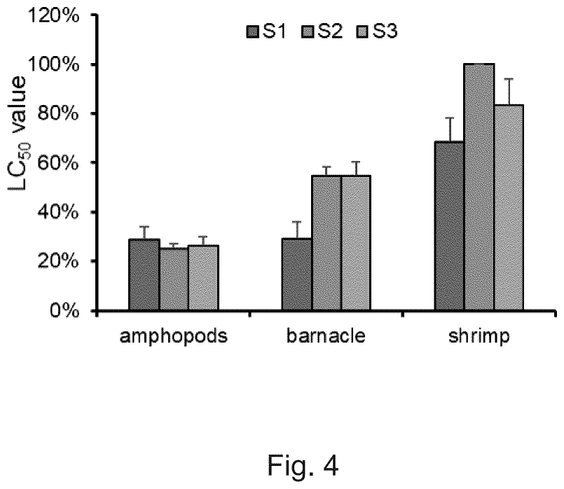

The salinity of sewage was adjusted to 30% % before the testings. Survival rates of different animals after 48-h exposure of three sewage effluents samples are shown in FIG. 4. Among the testing animals, the amphipods were the most sensitive animal, followed by barnacle larvae and shrimps. Similarly, previous study also showed that amphipods were generally sensitive to sewage effluents. The survival rates of all animals were slightly lower under S2 and S3 exposure, which were probably due to the higher metal concentrations in these two sewage effluent samples. LC50s of effluents determined were 25.0˜28.8%, 29.0˜54.6% and 68.4˜83.3% for amphipods, barnacle larvae and shrimps, respectively (FIG. 4).

### Example 2

### Cell Viability under Effluent Exposure

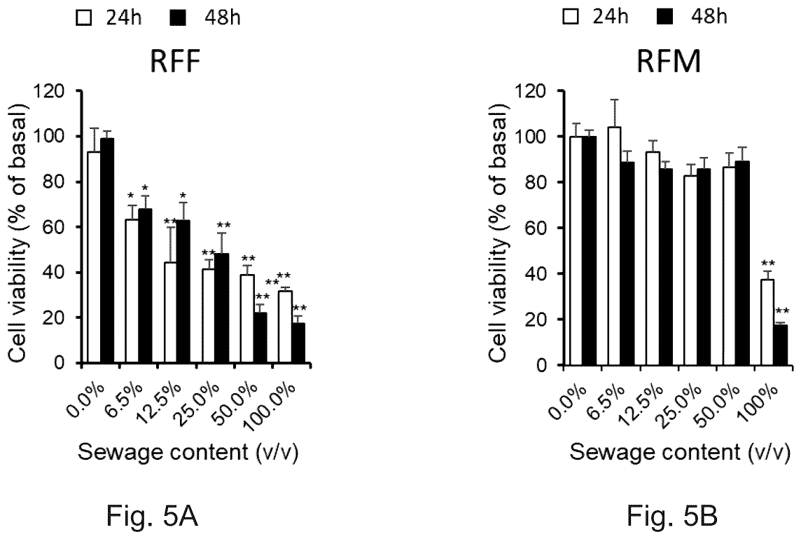

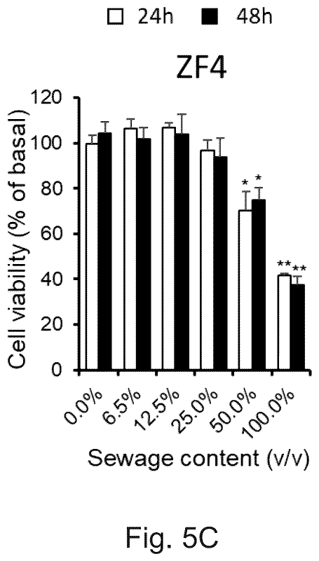

The cytotoxicity of sewage effluent S1 on RFF cells and other two fish cell lines including a macrophage from rabbitfish (RFM) and a liver cell line from zebrafish (ZF4) were tested and shown in FIGS. 5A to 5C. Here, different concentrations of sewage effluents (6.5%, 12.5%, 25%, 50% and 100% v/v) were added into the medium and cultured for 24 h and 48 h, respectively. Saltwater with the same salinity of sewage effluents sample was used as a negative control. Meanwhile, a significant decrease of cell viability occurred when the sewage effluents was added at the concentration of 6.5%. The cell viability of RFM and ZF4 cell showed significant decrease only when the sewage effluents content reached about 100%, which indicated that the RFF showed a most sensitive response to sewage effluents exposure (FIG. 5A). The results showed that RFF cell line represented sewage effluents content accurately and sensitively under exposure.

### Example 3

### Cellular Responses under Sewage Effluents Exposure

To further investigate the ‘structure’ of cellular organelles, several probes were applied to visualize the subcellular changes. Among cellular organelles, it is believed that mitochondria act as the powerhouses of cell regulated the tricarboxylic acid cycle, oxidative phosphorylation (OXPHOS), and ATP production, providing energy for cell survival and functions. This organelle hosts important biosynthetic and bioenergetic progress in cells, and not only controls cell proliferation, differentiation and death, but also contributes to immune response of the body. This organelle is also responsible for transporting different metal cations, including Ca²⁺, K⁺, Na⁺, Mg²⁺, Zn²⁺, which is essential for maintaining metal homeostasis in cells. Impairment of metal homeostasis leads to mitochondrial dysfunction.

Apart from mitochondria, lysosomes as the most essential organelle were investigated as well. The main structure of a lysosome is an acid lumen which contains a host of hydrolytic enzymes, including nucleases, proteases, phosphatases, lipases, sulfatases. This organelle constitutes the main sites of toxic metal and organic pollutant sequestration and detoxification. The responses of this organelle have been proved as a sensitive stress index in responding to metal pollution. According to these characteristics, lysosomal changes received much attention as potential ‘early warning tools’ for the estimation of the biological effects of pollutants. Changes in lysosomes including their size and pH were thus monitored.

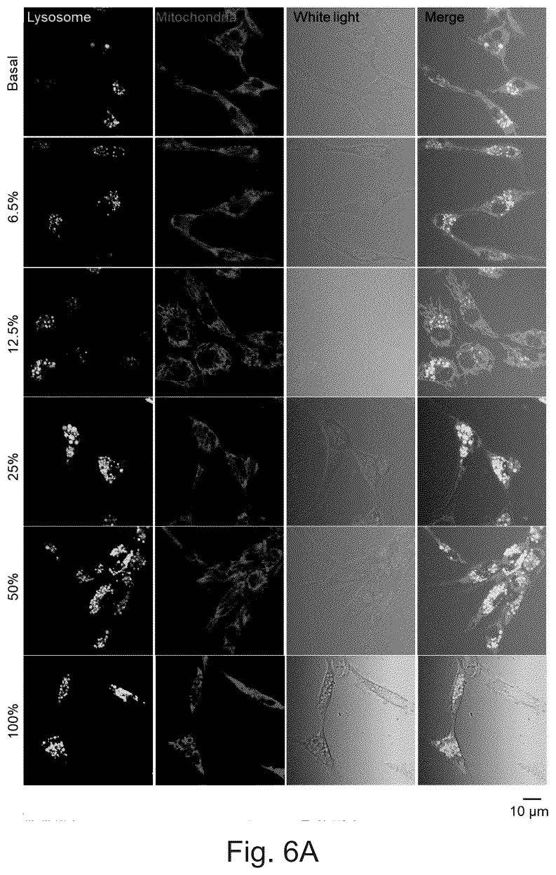

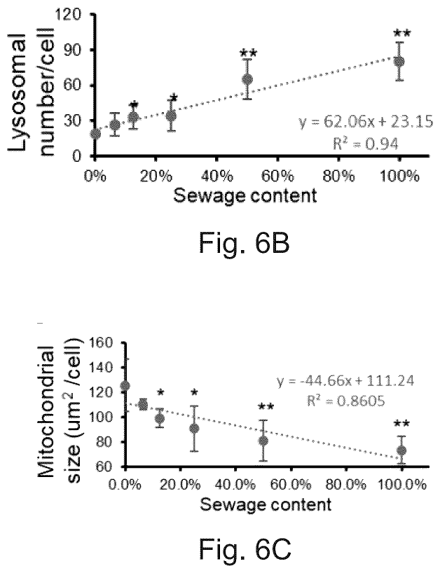

RFF cells were exposed to different concentrations of sewage effluents for 24 h. Then the cells were stained with fluorescent probes to visualize the structure of lysosomes and mitochondria. Confocal graphs were taken (FIG. 6A) and the lysosomal number and mitochondrial size in each cell were quantified based on the confocal images. Under sewage effluents exposure, the lysosomal number increased from ˜20 to ˜60 in each cell (FIG. 6B). Meanwhile the lysosomal number significantly increased at the sewage effluent content of 12.5%. When compared the lysosomal number with sewage effluents concentration, the lysosomal number was linearly related to sewage effluents content (R²⁼0.93) (FIG. 6B). The increased lysosomal number was a cellular response in digesting pollutants which is in line with the muscle cell under Cd and Hg exposure.

Due to their susceptibility to damage, mitochondria are highly sensitive to environmental toxicants. Under sewage effluent exposure, the mitochondrial size decreased from ˜125 to ˜73 μm2 in each cell. A significant decrease was observed when the sewage effluents content reached 12.5%. In addition, the decrease of mitochondrial size was linearly related to sewage effluent concentration (FIG. 6C). This subcellular response was similar to the liver cell under small particles exposure. The shrink of mitochondrial size was related to the dysfunction of this organelle which led to cell death afterwards. Thus, under sewage effluent exposure, more lysosomes were formed, and the mitochondria were shrined in RFF cells. According to these results, lysosomal number and mitochondrial size could be used as two parameters in representing the subcellular effects of sewage effluents.

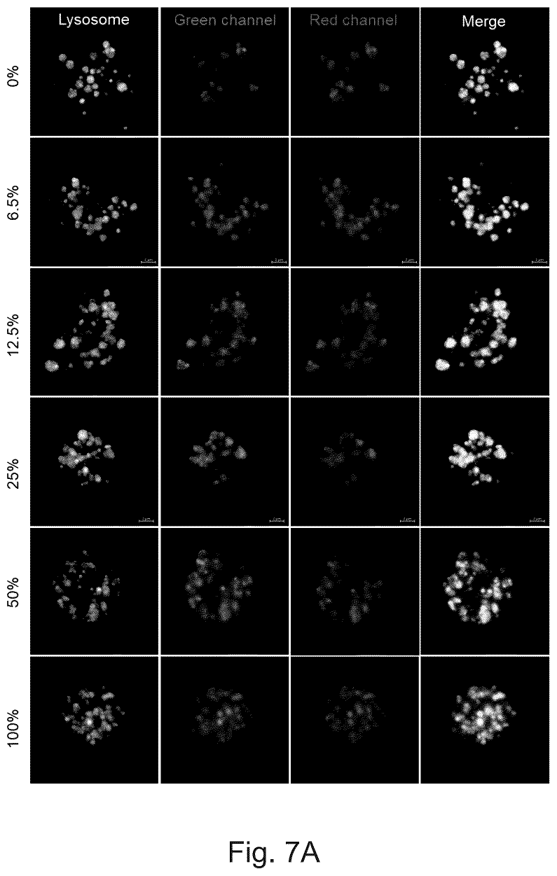

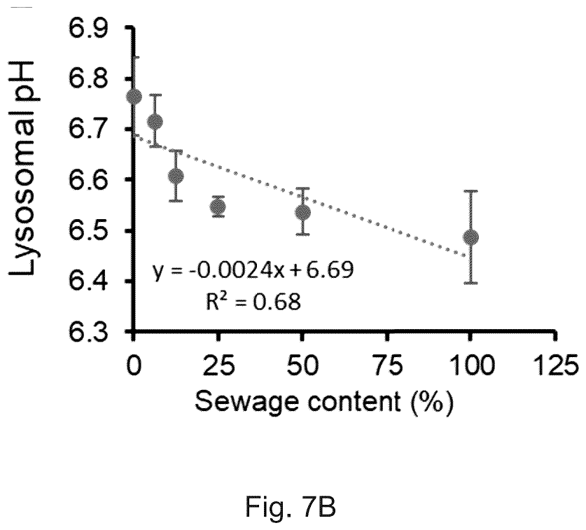

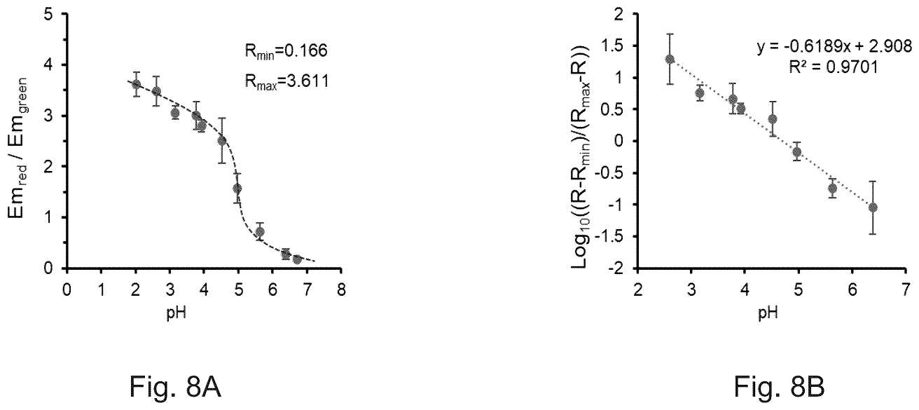

To further investigate the changes in lysosomal function under sewage effluent exposure, the lysosomal pH was stained and measured as well (FIGS. 7A and 7B). Here a specific probe CSMPP was used to stain the cell after sewage effluents exposure. The fluorescent intensity of green channel and red channel was used to calculate the pH value (FIGS. 8A and 8B). Under sewage effluent exposure, lysosomes were acidified, and the pH value decreased from 6.8 to 6.5. Additionally, the pH decreased linearly with increasing sewage effluents concentration (FIG. 7B). The digestive enzymes inside lysosomes required a low pH to be active. The decreased pH further proved the digestion process of lysosome under the exposure of sewage effluents. This result indicated that lysosomal pH could be used to represent the effect of sewage effluents on RFF cells.

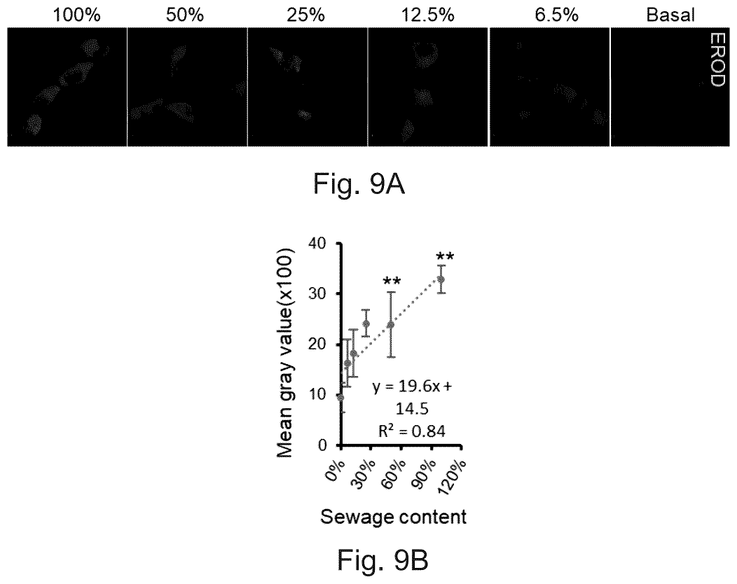

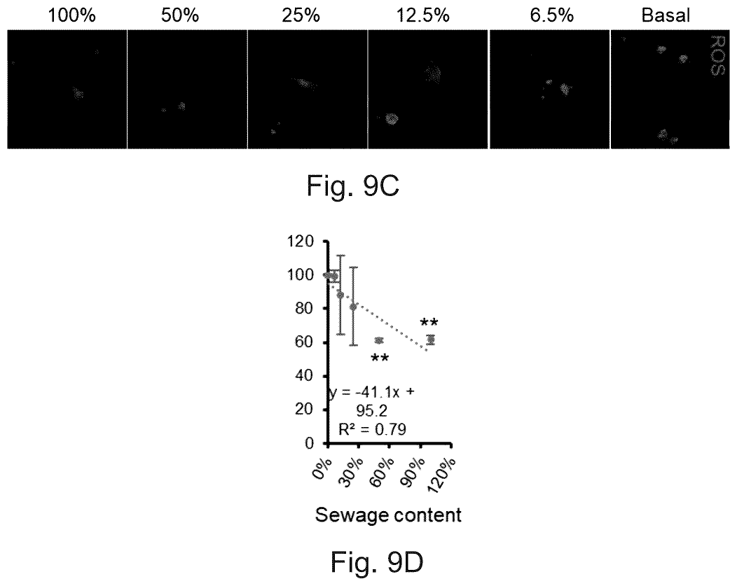

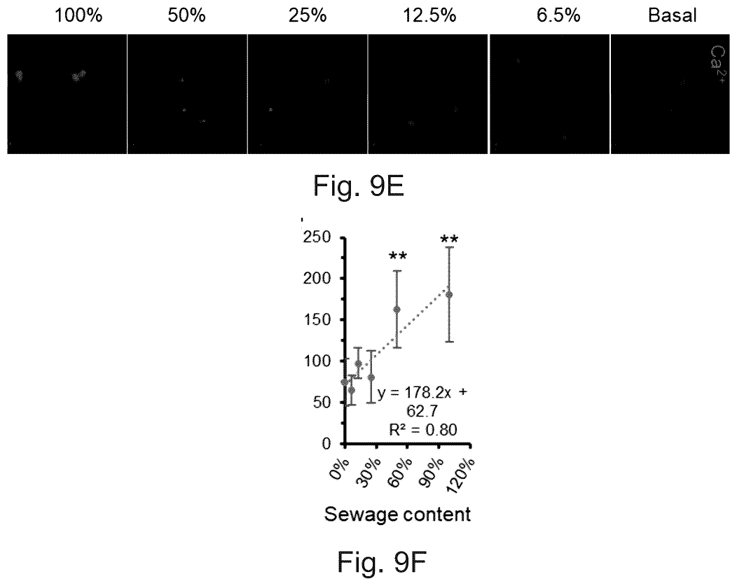

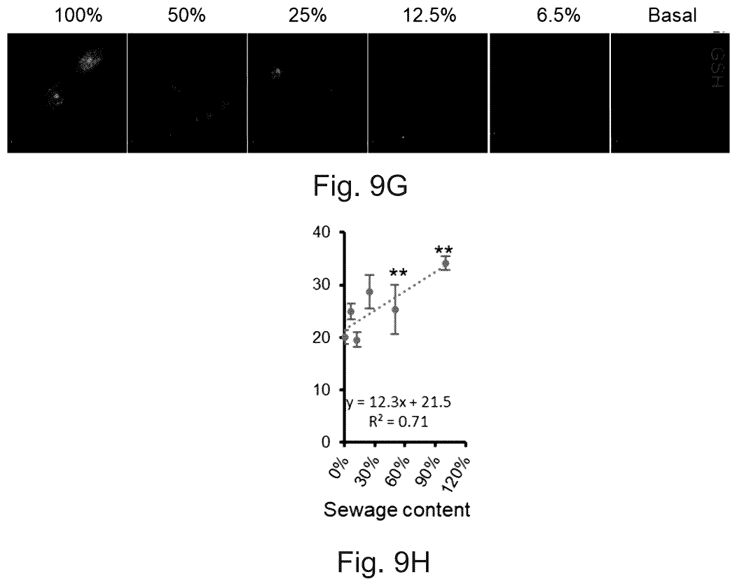

Apart from investigating the changes in cell organelles, some enzymes related to cellular functions and detoxification including EROD activity (FIGS. 9A and 9B), ROS production (FIGS. 9C and 9D), calcium (Ca²⁺) influx (FIGS. 9E and 9F), glutathione (GSH) formation (FIGS. 9G and 9H) were visualized and measured. It is believed that the induction of 7-ethoxyresorufin-O-deethylase (EROD) activity is a valuable biomarker in ecotoxicological studies. EROD activities were highly related to the concentrations of organic contaminants. In the cell-based testing, the activity of EROD increased with sewage effluent concentration (FIG. 9D). One possible explanation was that the increase of EROD was mainly related to the organic pollutants in sewage effluents. The increasing cellular EROD was in line with the rainbow trout and mirror carp under wastewater exposure.

It is appreciated that ROS is a double-edged sword in cell metabolism. At low to moderate levels, it acts as signal transducers to activate the cell proliferation, migration, invasion, and angiogenesis. In contrast, high levels of ROS would cause damage and led to cell death. The production of ROS decreased with sewage effluent concentration (FIGS. 9C and 9D). The decrease of ROS formation was probably caused by the shrink of mitochondria (FIG. 6C), mainly because about 90% of ROS was generated from the mitochondria.

Intracellular Ca²⁺ concentration played major roles in cell cycle progression, cell growth, proliferation and division. It is believed that Ca²⁺ signaling was related to persistent organic pollutants and metal pollutants. For this reason, the Ca²⁺ influx was determined under sewage effluents exposure (FIG. 9E). The intracellular Ca²⁺ was found to dose-dependently increase with the sewage effluents content (FIG. 9F). It is believed that the increase of Ca²⁺ was mainly related to the dysfunction of cells under sewage effluents exposure.

GSH plays a key role in regulating the redox homoeostasis and metal homoeostasis. The GSH formation increased with sewage effluents concentration (FIG. 9H), which was probably related to the metal pollutants in sewage effluents. This reaction was similar to the catfish under oxidative stress and metal and Eurasian carp under metal exposure.

All these four indexes (EROD activity, ROS production, Ca²⁺ influx, and GSH formation) were linearly related to sewage effluents concentrations which indicated the potential in representing sewage effluents toxicity. However, significant differences only occurred when the sewage effluents content reached about 50%. The sensitivity of these four cellular indexes were not as high as the lysosome and mitochondria.

To further simplify the facility required in cell-based testing, the fluorescence plate reader was applied to replace the usage of confocal microscope in fluorescence intensity quantification. Here, fluorescence intensities of previous cellular parameters in RFF cells were measured and normalized by cell number after sewage effluents exposure. Then the normalized fluorescence values were compared with the one without sewage effluents addition to achieve the folds of changes in cellular parameters under sewage effluents exposure.

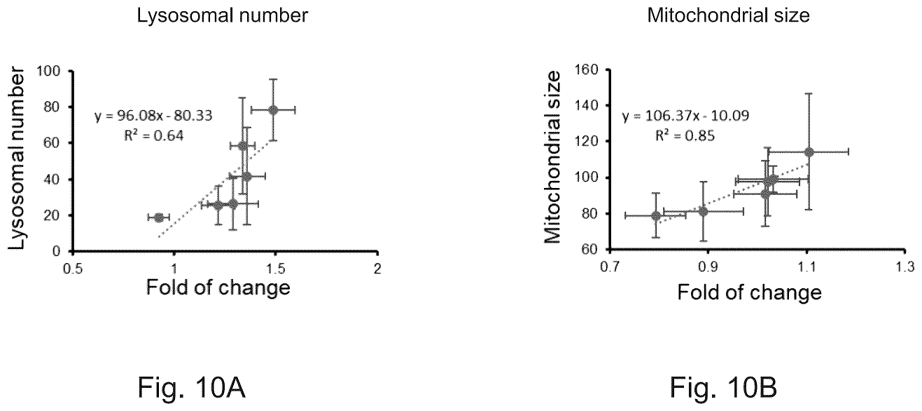

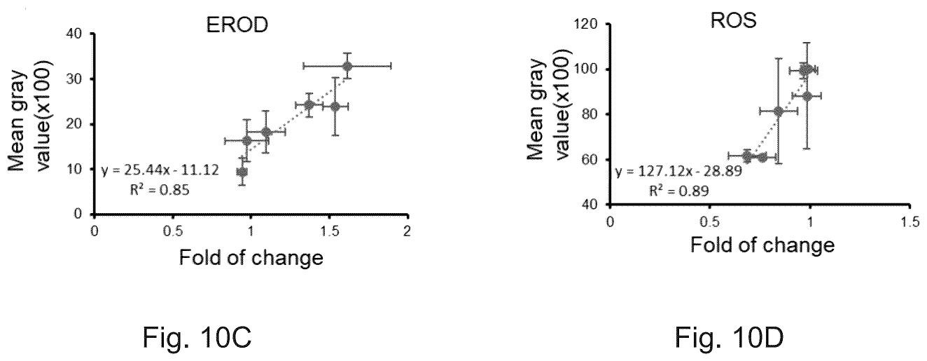

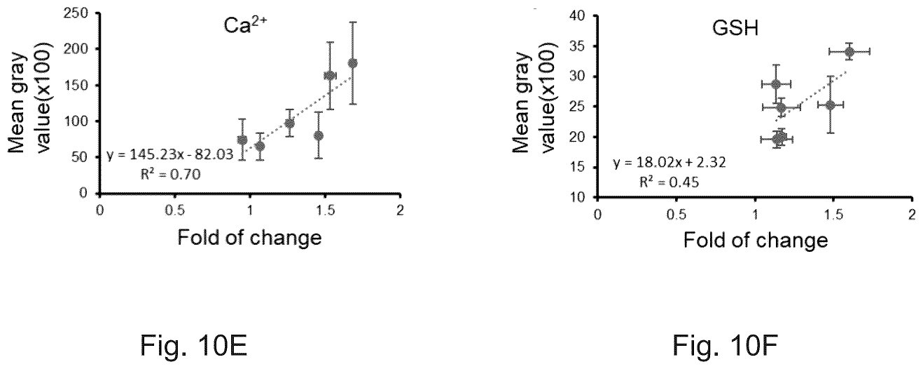

To confirm the accuracy of result from plate reader, each cellular parameter was compared with the fold of changes in fluorescence intensity separately (FIGS. 10A to 10F). According to the calibration curves, mitochondrial size, EROD activity, ROS formation, and Ca²⁺ influx showed a linear relationship between graphical results and plate reader's results (R^{2>0.8}) (FIGS. 10B to 10E), whereas the lysosomal number and GSH content gained from the confocal microscope were not in line with the plate reader (R^{2=0.64} and 0.45) (FIGS. 10A and 10F).

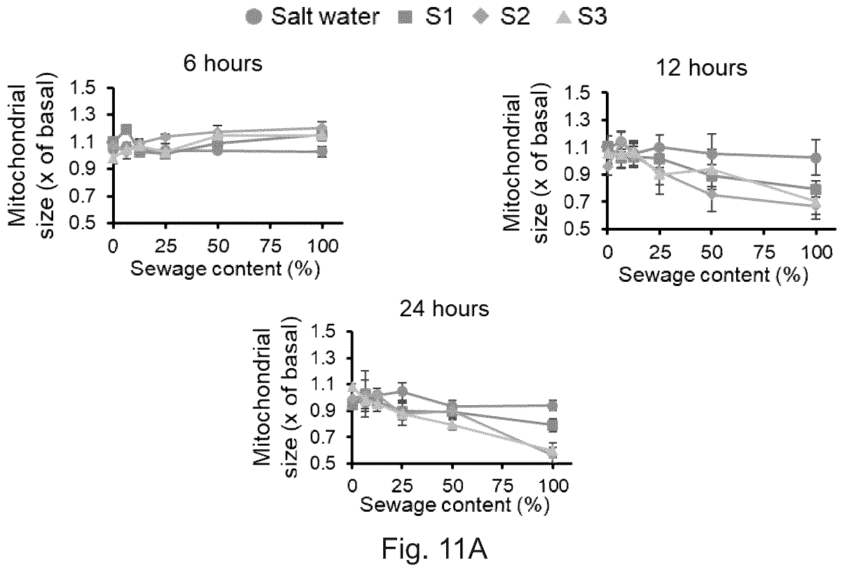

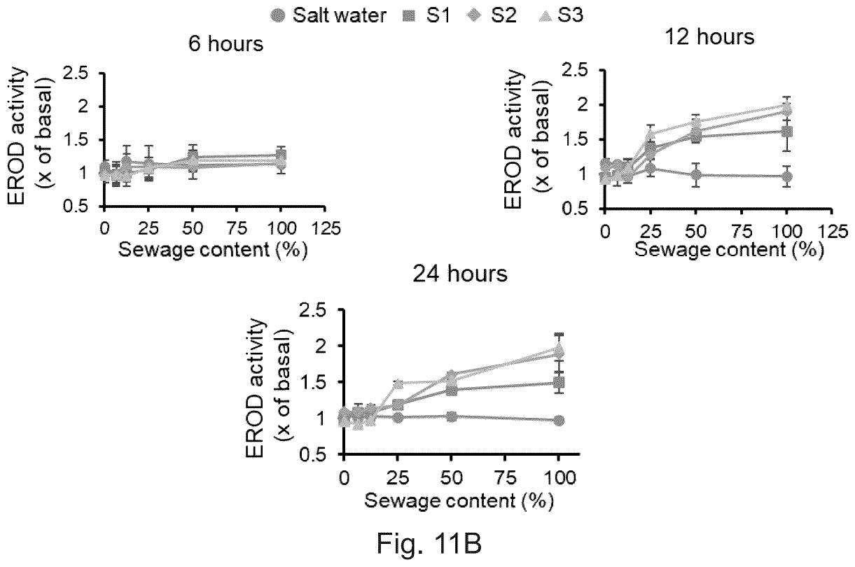

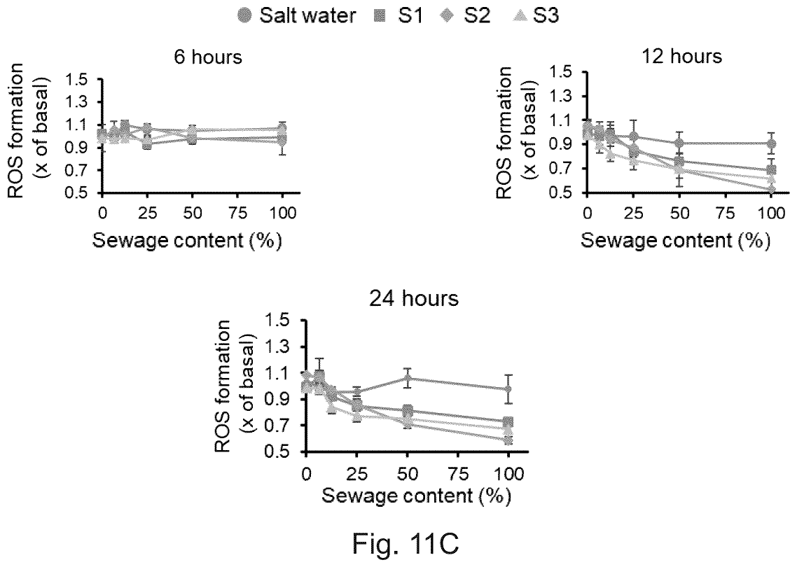

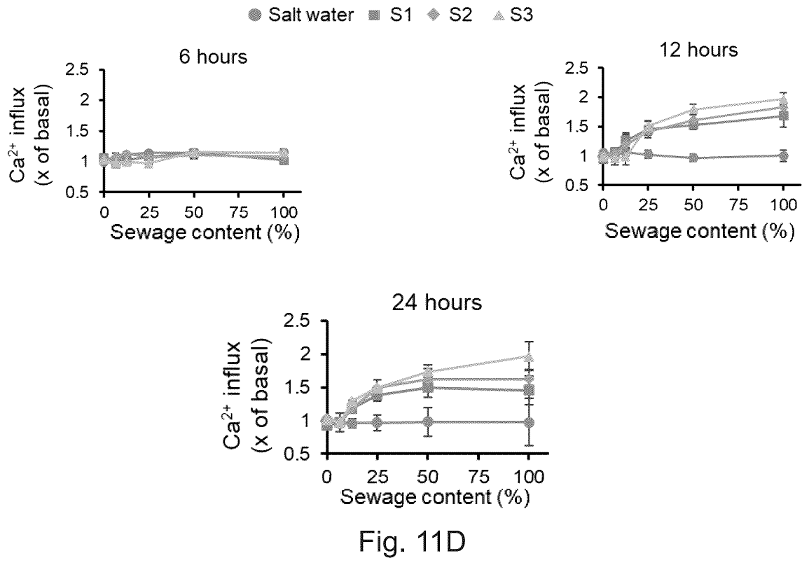

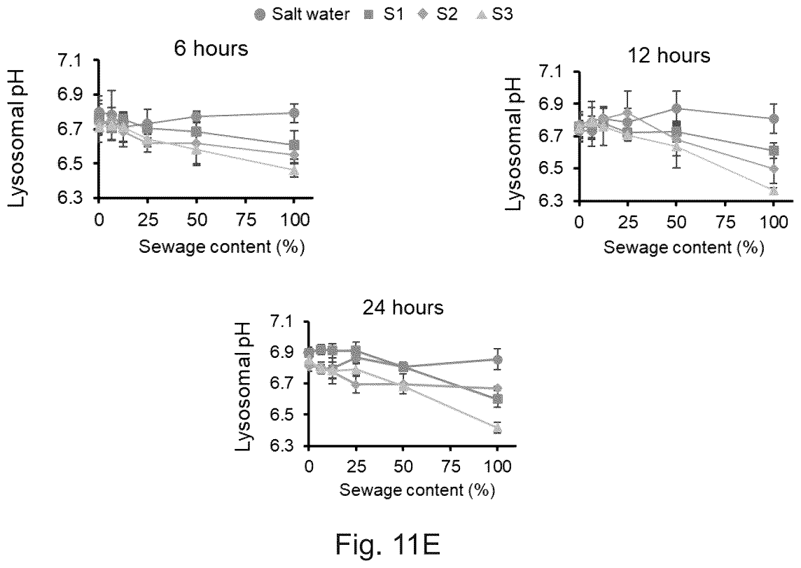

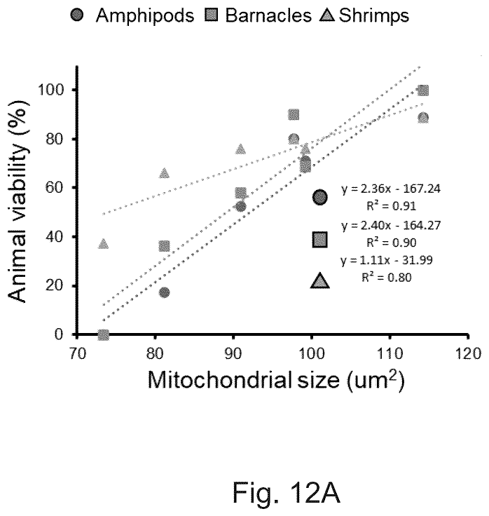

These results indicated that the plate reader could partly replace the usage of confocal microscope in this cell-based testing. Additionally, attempt has been made on reducing the exposure duration. RFF cells were exposed to the three sewage effluents samples (S1, S2, S3) with different exposure periods (6 h, 12 h, 24 h) (FIGS. 11A to 11E). Afterwards, fluorescence values of cellular parameters were tested by plate reader as described above. Similar trends were observed in folds of changes in fluorescence intensity. The cellular indexes showed no change under 6-h exposure except the lysosomal pH. Significant changes were found in the sewage effluents content of 12.5% under 12 h exposure when compared with the saltwater at the same salinity (FIGS. 11A to 11E). Meanwhile the fold changes in cellular indexes showed no significant difference between 12-h and 24-h exposure treatments. This could help shorten the time and simplify the facilities required in toxicity determination.

### Example 4

### Relationship between Cellular Indexes and Animal Survival Rate

To further confirm that the subcellular indexes could represent the acute toxicity on animals, cellular parameters gained by confocal microscope and fluorescence plate reader were compared with the toxicity of testing animals (amphipods, barnacles, and shrimps) at the same sewage effluents contents.

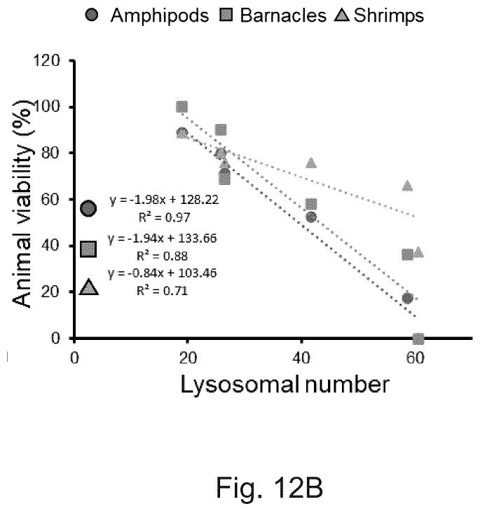

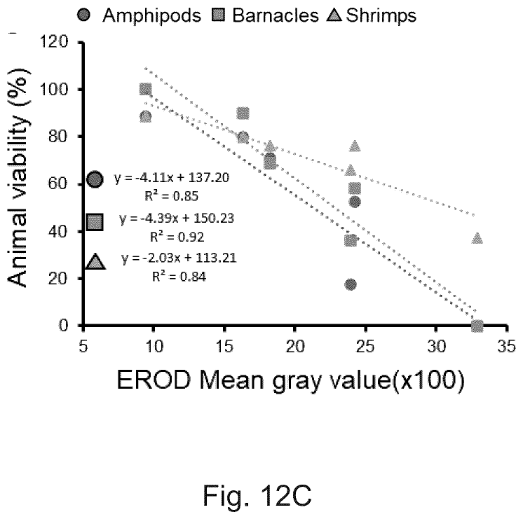

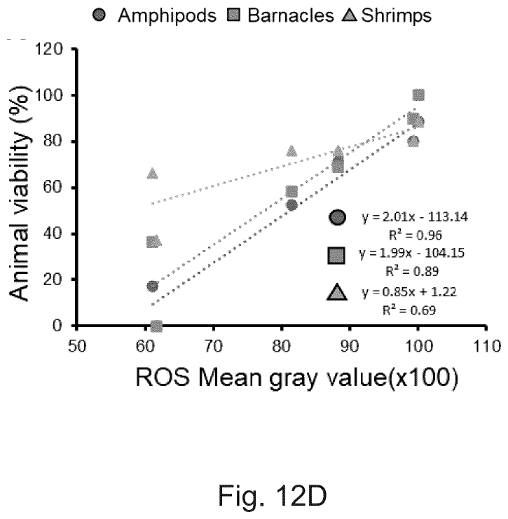

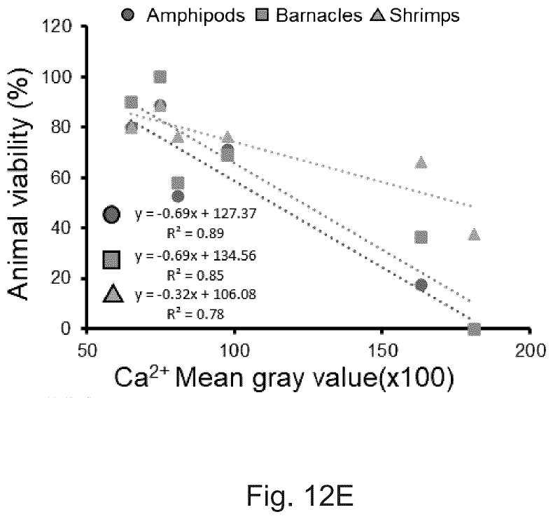

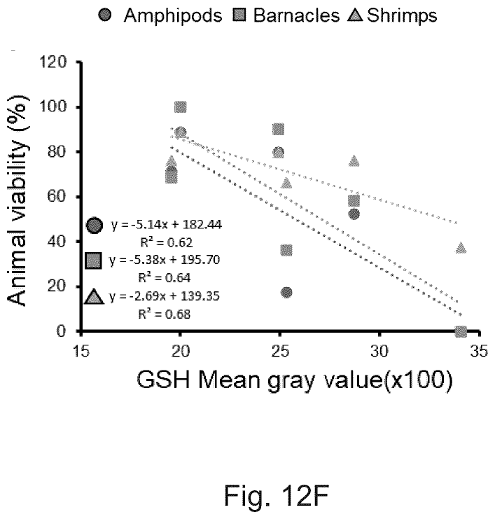

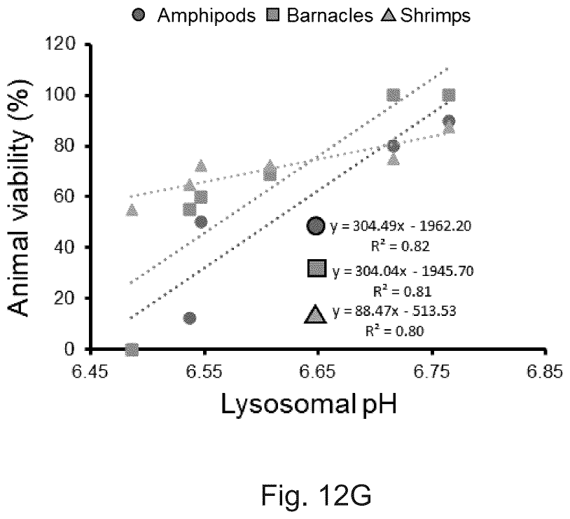

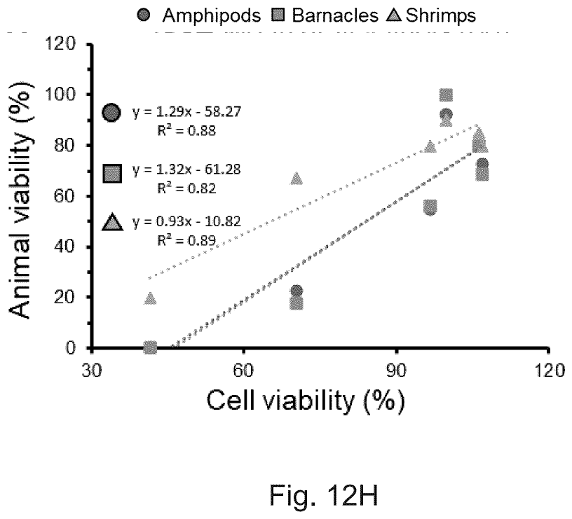

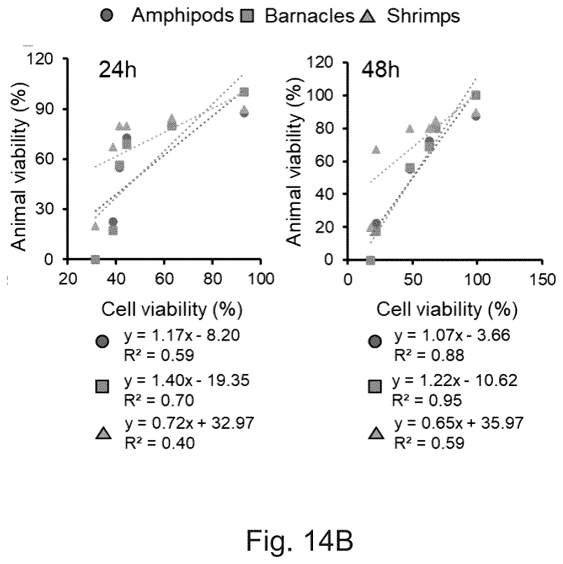

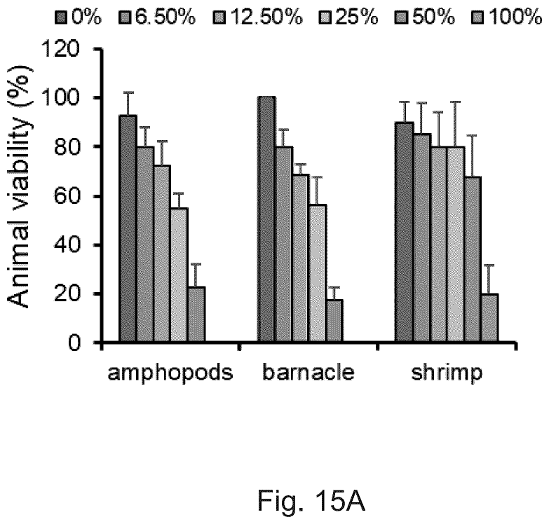

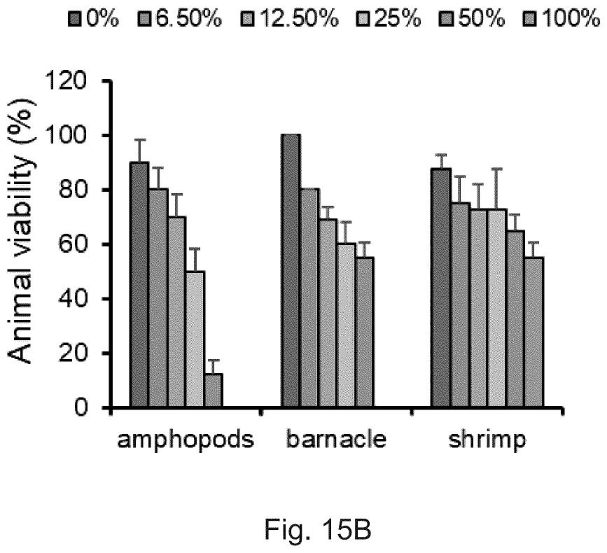

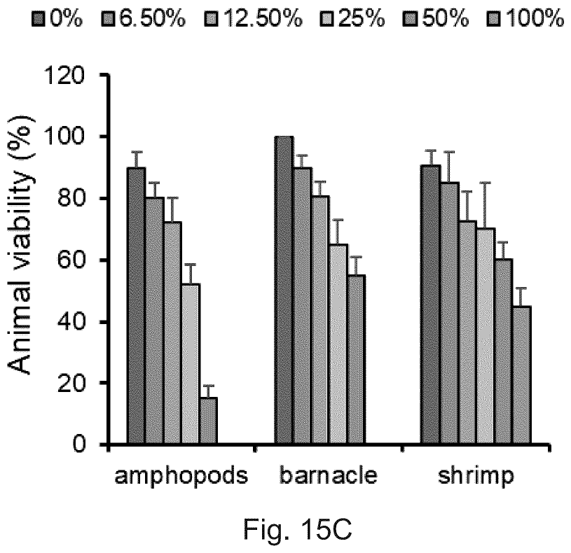

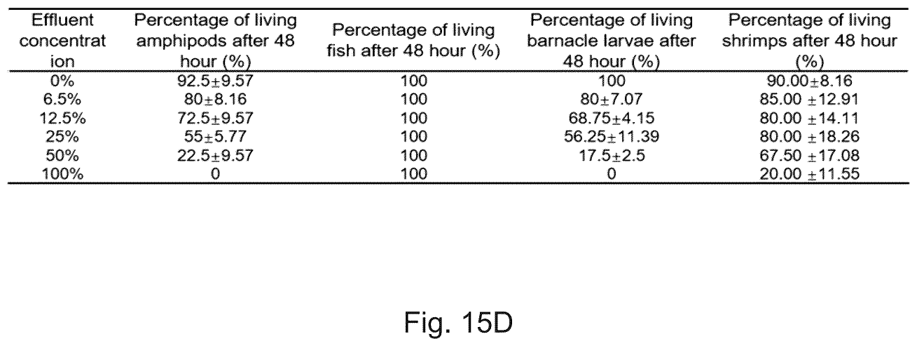

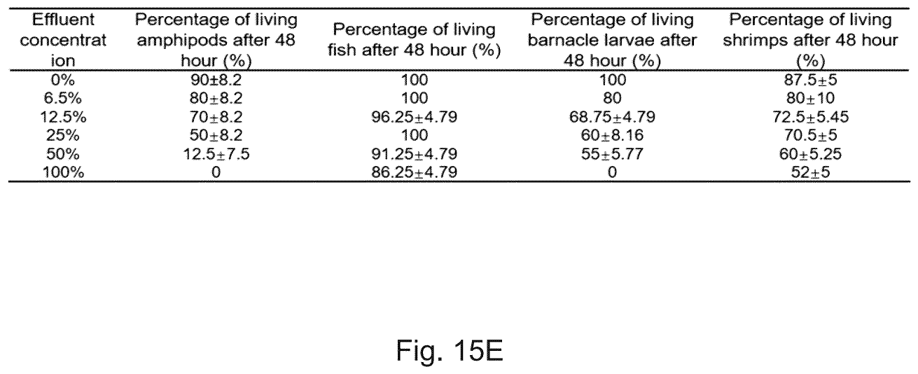

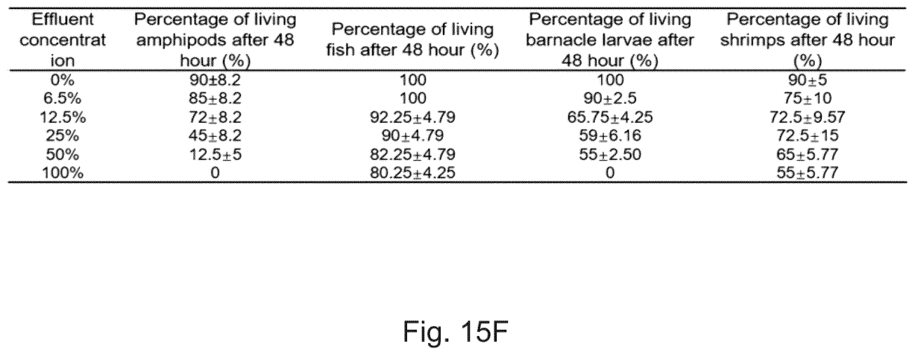

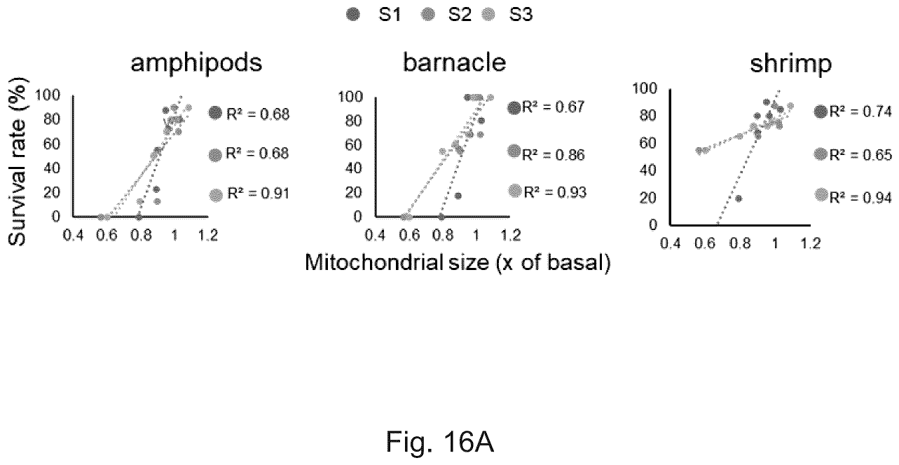

All cellular indexes except GSH content showed a linear relationship with the viability of amphipods and barnacles (R²>0.8) (FIG. 12F). Since GSH might not affect the animal survival directly, the GSH content was not linearly related with the viability of all three testing animals and was not applied for further toxicity determination. The R² of cellular indexes with shrimp viability were not as high as the other two animals. Only mitochondrial size, EROD activity and lysosomal pH showed a good linear relationship with the shrimp's viability (FIGS. 12A, 12C and 12G). This indicated that the acute toxicity result of amphipod, barnacle and shrimp could be represented by these cellular indexes. Amphipods and barnacle larvae were considered as the sensitive model animals in toxicity determination and water quality assessment. Compared with the subcellular parameters, the cell viability did not show a good linear relationship with the sewage effluents content (R^{2<}0.8), which further suggested the inaccuracy in toxicity determination using cell viability index alone (FIG. 14A). By plotting the cell viability against the animal survival rate (FIGS. 12H, 14B, and FIGS. 15A to 15F), the cell viability of RFF cell showed a poor linear relationship with the animal survival rate under the same exposure concentration (R^{2˜}0.5). This result indicated that the cell viability was not able to represent the animal survival under sewage effluents exposure.

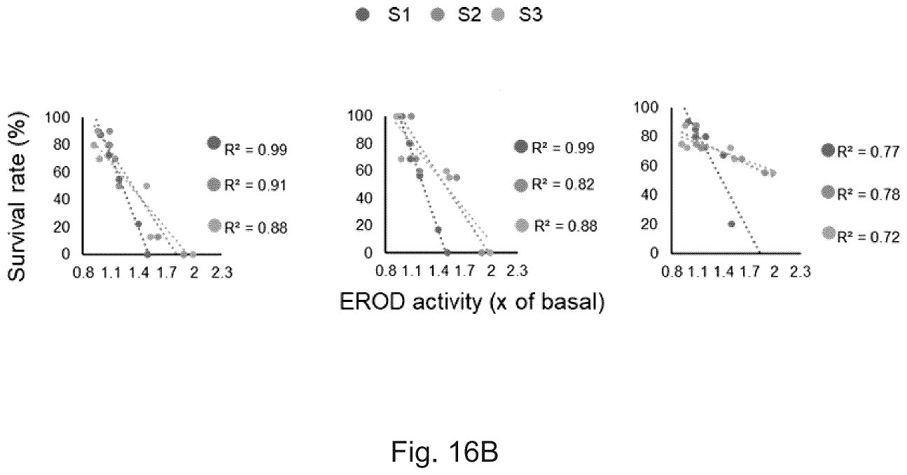

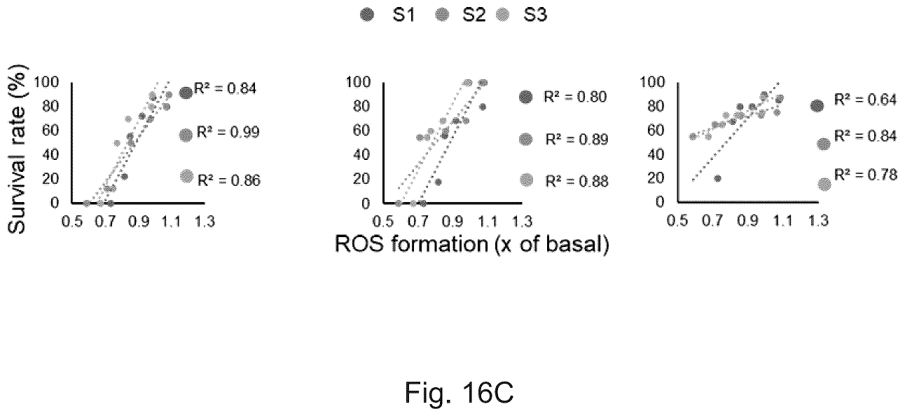

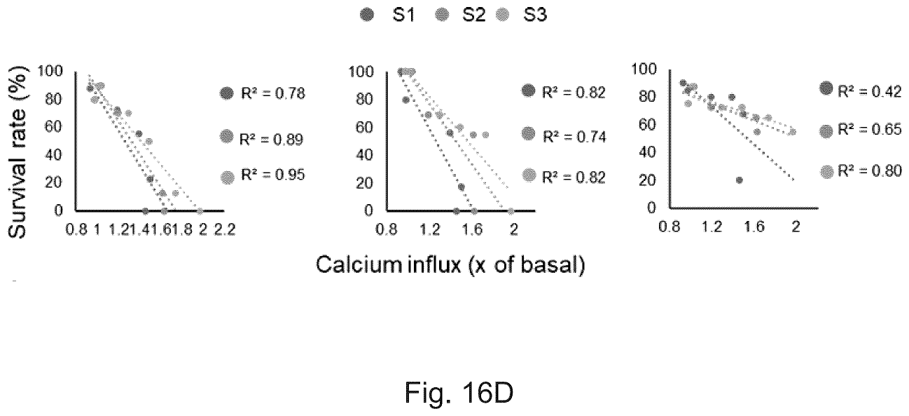

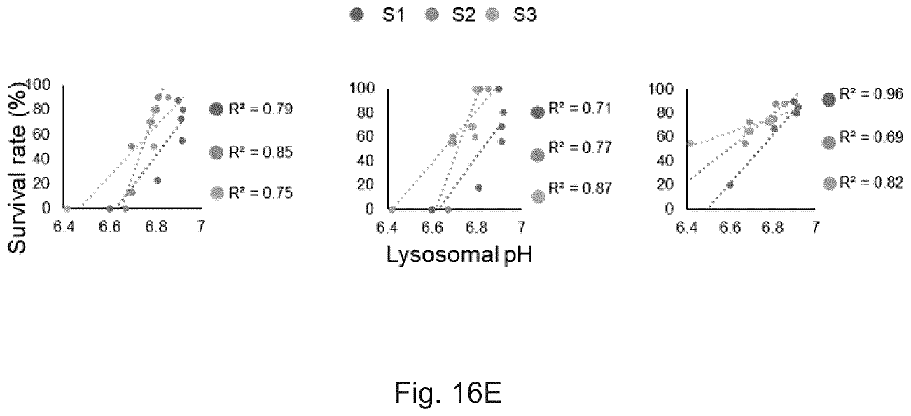

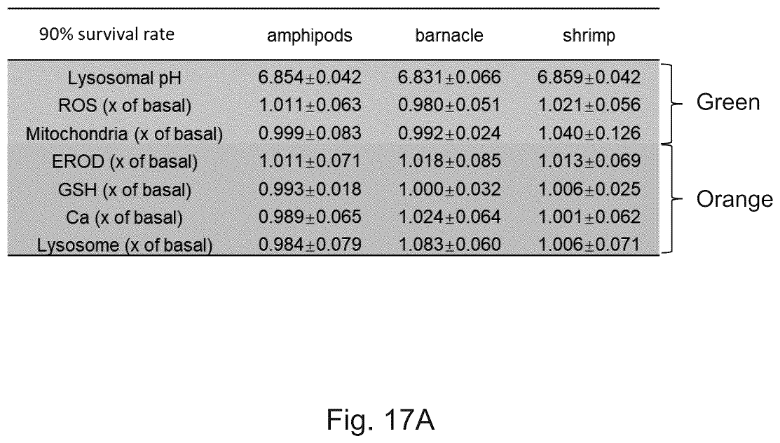

To confirm the accuracy of results based on the 12-h exposure, the changes in cellular indexes (as obtained by fluorescence plate reader) were compared with the animal testing results (FIG. 16). These cellular indexes similarly showed a good linear relationship with the amphipod and barnacle results while the relationships with shrimps were poor. These results indicated that the cell-based testing could be operated by fluorescence plate reader and 12-h exposure duration could reflect the sewage effluents toxicity. In general, subcellular changes could partly represent the in-vivo acute toxicity of sewage effluents, especially the animals very sensitive to pollutant.

### Example 5

### Toxicity Determination using the Calibrated Cellular Parameters and Animal Viability

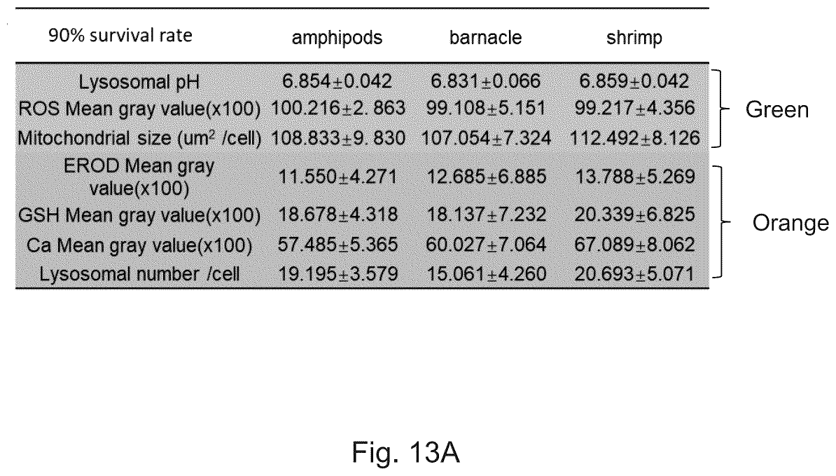

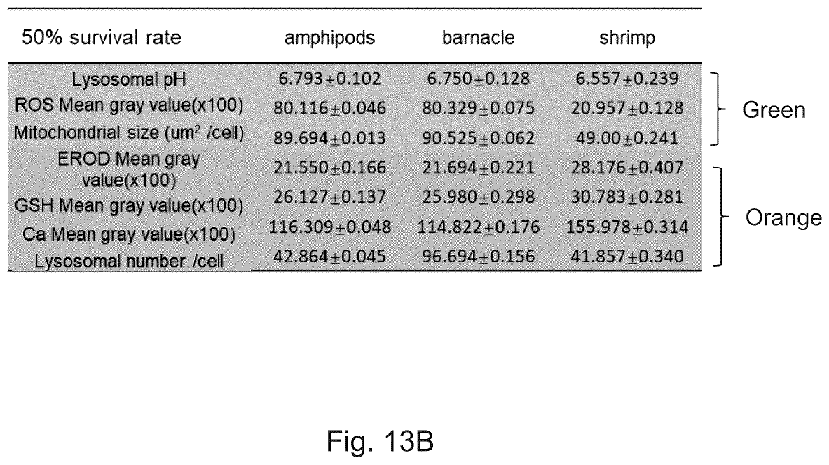

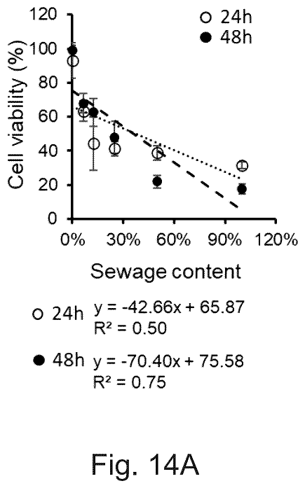

After the calibration of cellular parameters with animal viability (i.e. establishing a linear regression relationship model between the cellular parameters and the animal viability), the value of cellular parameters upon the death rate of animal reached at 10% (LC₁₀) and 50% (LC₅₀) could be calculated using the linear regression equations of the model. For example, when the cellular parameters were obtained by way of confocal microscopy, a set of linear regression relationship models as illustrated in FIGS. 12A to 12G may be established. Then, the value of cellular parameters upon the death rate of animal reached at 10% (LC₁₀) and 50% (LC₅₀) could be calculated using the linear regression equations as shown in FIGS. 12A to 12G.). The value of each cellular parameter of each animal was listed in the tables as shown in FIGS. 13A and 13B. As such, it is believed that one may consider that the values of cellular parameters shown in the tables (FIGS. 13A and 13B) as a standard in determining (acute) toxicity of sewage samples (LC₁₀ or LC₅₀).

To determine the acute toxicity of an unknown sewage effluent sample, firstly, the fluorescent probe-stained RFF cells may be incubated with the sewage effluent sample. Given that it is evident from the above examples that the incubation time of 12 hours and 24 hours would have insignificant effect on the obtained cellular parameters, the stained RFF cells may be incubated for 12 hours or optionally 24 hours depending on actual needs. In this example, the stained RFF cells may be incubated with the sewage effluent sample for 24 hours. Then, one or more cellular parameters from the RFF cells exposed with sewage effluent is recorded by confocal microscope and quantified. After that, this cellular parameter(s) may be compared with the one in FIGS. 13A and 13B for acute toxicity determination.

If the tested value is higher than the one in orange area or lower than the one in green area as shown in FIGS. 13A and 13B, it would indicate that this sewage effluent sample could cause 10% or 50% death of certain animal, and such sample would be considered as toxic. It also provides an indication that treatment or dilution of such sample would be required to achieve the disposal standard. For example, assuming that the mitochondrial size of the RFF cells after exposing to the sewage effluent sample is determined to be 50 and the EROD value is determined to be 30, by comparing with the mitochondrial size and EROD data in FIG. 13B, it could be determined that this sewage effluent sample could cause 50% of death of amphipods, barnacle and shrimp. And this sewage effluent sample could be considered as toxic.

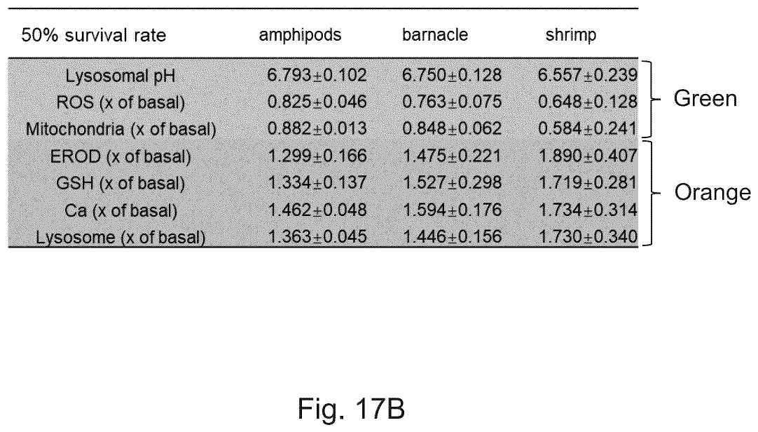

Alternatively, when the cellular parameters were obtained by way of a fluorescence plate reader, the value of cellular parameters upon the death rate of animal reached at 10% (LC₁₀) and 50% (LC₅₀) could be calculated using the linear regression relationship models and linear regression equations as shown in FIGS. 16A to 16F, and the values are shown in FIGS. 17A and 17B. To determine the acute toxicity of an unknown sewage effluent sample, similarly, the stained RFF cells may be incubated with the sewage effluent sample for, e.g., 12 hours, followed by recording one or more of cellular parameters from the RFF cells exposed with sewage effluent with a fluorescence plate reader, and comparing this recorded cellular parameter(s) with the values shown in FIGS. 17A and 17B for acute toxicity determination. Similarly, if the tested value is higher than the one in orange area or lower than the one in green area as shown in FIGS. 17A and 17B, it would indicate that this sewage effluent sample could cause 10% or 50% death of certain animal, and such sample would be considered as toxic.

The invention has been given by way of example only, and various other modifications of and/or alterations to the described embodiment may be made by persons skilled in the art without departing from the scope of the invention as specified in the appended claims.

1. A method for determining acute toxicity of an analyte, comprising the steps of:
a) exposing a first fish cell culture to a first reference solution;
b) incubating the first fish cell culture in step a) with a fluorescent probe;
c) obtaining a first fluorescence parameter from the stained first fish cell culture;
d) establishing a first linear regression relationship model between the first fluorescence parameter and viability of an animal model; and
e) determining acute toxicity of the analyte by adopting the first linear regression relationship model.

2. The method as claimed in claim 1, wherein the first fish cell culture comprises a fish fin cell line isolated from grey rabbit fish (Siganus fuscescens).

3. The method as claimed in claim 1, wherein step a) comprises the step of collecting the first reference solution from a reference site at a first time point.

4. The method as claimed in claim 3, wherein the first reference solution comprises a 24-h flow-weighted composite effluent from a sewage effluent treatment work.

5. The method as claimed in claim 1, wherein the first reference solution has a concentration of pollutant by volume selected from any one of 0%, 6.5%, 12.5%, 25%, 50%, and 100%.

6. The method as claimed in claim 1, wherein step b) comprises the step of incubating a mixture of the first fish cell culture and the fluorescent probe in the dark.

7. The method as claimed in claim 1, wherein the fluorescent probe is selected from the group consisting of lysosomal tracker, mitochondrial tracker, (Z)-3-(4-(4-methylpiperazin-1-yl)phenyl)-2-(4-(pyridin-4-yl)phenyl) acrylonitrile (CSMPP), 7-ethoxyresorufin, H₂DCFDA, Fluo-4, AM, ThiolTracker, and a combination thereof.

8. The method as claimed in claim 1, wherein step c) comprises the steps of:
taking confocal microscopy images of the stained first fish cell culture; and
obtaining the first fluorescence parameter of the stained first fish cell culture from the confocal microscopy images.

9. The method as claimed in claim 1, wherein step c) further comprises step c1) obtaining fluorescence intensity of the stained first fish cell culture by way of a fluorescence plate reader.

10. The method as claimed in claim 1, wherein the fluorescence parameter corresponds to the fluorescent probe and is selected from the group consisting of lysosomal number, mitochondrial size, lysosomal pH, EROD activity, ROS production, Ca²⁺ influx, GSH formation and a combination thereof.

11. The method as claimed in claim 1, wherein step d) comprises the steps of:
incubating the animal model with the first reference solution for at least 12 h;
determining the viability of the animal model; and
building a first linear regression equation between the first fluorescence parameter and the viability of the animal model and obtaining an R-squared (R²) value therefrom.

12. The method as claimed in claim 1, wherein the animal model comprises any one of amphipod (Melita longidactyla), barnacle larvae (Balanus amphitrite), and shrimp (Metapenaeus ensis).

13. The method as claimed in claim 11, wherein the first reference solution has a concentration of pollutant by volume selected from any one of 0%, 6.5%, 12.5%, 25%, 50%, and 100%.

14. The method as claimed in claim 11, wherein the R-squared value is at least about 0.70.

15. The method as claimed in claim 1, further comprising the steps of:
a′) exposing a second fish cell culture to the analyte;
b′) incubating the second fish cell culture in step a′) with a fluorescent probe; and
c′) obtaining a second fluorescence parameter from the stained second fish cell culture.

16. The method as claimed in claim 15, wherein step e) comprises the steps of:
determining, from the first linear regression relationship model, a reference value at 90% viability; and
comparing the second fluorescence parameter with the reference value at 90% viability to determine the acute toxicity of the analyte.

17. The method as claimed in claim 15, wherein step e) further comprises the steps of:
determining, from the first linear regression relationship model, a reference value at 50% viability; and
comparing the second fluorescence parameter with the reference value at 50% viability to determine the acute toxicity of the analyte.

18. The method as claimed in claim 1, wherein the analyte comprises sewage effluent.

19. The method as claimed in claim 3, wherein step a) is repeated using a second and a third reference solution from the reference site at a second and third time point respectively.

20. The method as claimed in claim 19, wherein the first, second, and third time points are each different by a three-month interval.

21. A system for determining acute toxicity of an analyte in accordance with the method as claimed in claim 1 comprising:
a first fish cell culture stained with a fluorescent probe;
a first reference solution in which the first fish cell culture is incubated; and
a first linear regression relationship model established from a first fluorescence parameter obtained from the stained first fish cell culture and viability of an animal model;
wherein the first linear regression relationship model contains acute toxicity of the analyte.
